(19) United States
(12) Patent Application Publication
(10) Pub. No.: US 2021/0212589 A1
(43) Pub. Date: Jul. 15, 2021

(54) SYSTEMS AND METHODS FOR MONITORING AN OBJECT

(71) Applicant: SHANGHAI UNITED IMAGING HEALTHCARE CO., LTD., Shanghai (CN)

(72) Inventors: Feng ZHANG, Shanghai (CN); Mingchao WANG, Shanghai (CN)

(73) Assignee: SHANGHAI UNITED IMAGING HEALTHCARE CO., LTD., Shanghai (CN)

(21) Appl. No.: 17/194,262

(22) Filed: Mar. 7, 2021

Related U.S. Application Data
(63) Continuation of application No. PCT/CN2019/104802, filed on Sep. 6, 2019.

(30) Foreign Application Priority Data
Sep. 7, 2018 (CN) 201811041745.7

(51) Int. Cl. A61B 5/055; A61B 5/00; A61B 6/03; G01R 33/28

(52) U.S. Cl. CPC A61B 5/055; G01R 33/281; A61B 6/032; A61B 5/0064

(57) ABSTRACT

A system and a method for monitoring an object may be provided. The system may include an optical assembly configured to acquire an optical signal associated with a region of interest (ROI) of a subject in an examination space of a medical device. The system may include an optical fiber bundle configured to transmit the optical signal. The system may include an optical sensing device configured to receive the optical signal and convert the optical signal to an electrical signal. The system may further include an image generator configured to generate, based on the electrical signal, a monitoring image of the ROI.

### CROSS-REFERENCE TO RELATED APPLICATIONS

This application is a Continuation of International Application No. PCT/CN2019/104802, filed on Sep. 6, 2019, which claims priority to Chinese Patent Application No. 201811041745.7, filed on Sep. 7, 2018, the contents of each of which are incorporated herein by reference.

### TECHNICAL FIELD

This disclosure generally relates to a medical system, and more particularly, to systems and methods for monitoring a status of an object.

### BACKGROUND

Various medical devices can be used to implement a medical diagnosis and/or treatment for a subject (e.g., a patient). Exemplary medical devices include a magnetic resonance imaging (MRI) device, a computed tomography (CT) device, a positron emission tomography (PET), a radiotherapy (RT) device, etc. Some medical devices include a bore for accommodating the subject to be scanned or treated. In some cases, when the subject is placed in the bore (e.g., in an internal examination space of the bore), it is difficult to observe the status of the subject directly. The status (e.g., a motion) of the subject can affect a scanning or treatment quality. For example, if the subject moves during a scan, a motion artifact can be introduced to a scanning image. As another example, if the subject feels uncomfortable (e.g., nausea, vomit, suffocation, etc.) during the scan, it is necessary to take steps to help the subject in time. Some existed electronic monitoring devices (e.g., a commercial camera) can be used to monitor the status of the subject during the scan. However, they can generate signal interference during operation. In addition, these electronic monitoring devices generally include metallic materials, which can introduce a signal interference into the medical device(s). Therefore, it is desirable to develop systems and methods that are configured to monitor the subject efficiently, and have a good compatibility with the medical device.

### SUMMARY

In a first aspect of the present disclosure, a system is provided. The system may include an optical assembly, an optical fiber bundle, an optical sensing device and an image generator. The optical assembly may be configured to acquire an optical signal associated with a region of interest (ROI) of a subject in an examination space of a medical device. The optical fiber bundle may be operably connected to the optical assembly, and configured to transmit the optical signal. The optical sensing device may be operably connected to the optical fiber bundle, and configured to receive the optical signal and convert the optical signal to an electrical signal. The image generator may be operably connected to the optical sensing device, and configured to generate, based on the electrical signal, a monitoring image of the ROI.

In some embodiments, the optical assembly may be operably located in the examination space of the medical device, or be movable in or to the examination space of the medical device.

In some embodiments, the optical sensing device and the image generator may be disposed outside the medical device. A first distance between the optical sensing device and the medical device may be greater than a first safety distance and a second distance between the image generator and the medical device may be greater than a second safety distance.

In some embodiments, the medical device may include at least one of: a magnetic resonance imaging (MRI) device, a computed tomography (CT) device, a radiotherapy (RT) device, a positron emission tomography (PET) device.

In some embodiments, the first safety distance may be set such that a working current of the optical sensing device has no effect on a working condition of the medical device, and the second safety distance may be set such that a working current of the image generator has no effect on a working condition of the medical device.

In some embodiments, a first beam splitter may be disposed between the optical fiber bundle and the optical sensing device, and configured to transmit the optical signal from the optical fiber bundle to the optical sensing device. A projector may be configured to convert a second electrical signal relating to a second image to a second optical signal, and transmit the second optical signal to the first beam splitter. The first beam splitter may be further configured to transmit the second optical signal to the optical fiber bundle.

In some embodiments, a second beam splitter may be disposed between the optical fiber bundle and the optical assembly. The second beam splitter may be configured to transmit the optical signal from the optical assembly to the optical fiber bundle, and transmit the second optical signal from the optical fiber bundle to a display region such that the second image relating to the second optical signal is presented on the display region.

In some embodiments, the display region may be in the examination space of the medical device.

In some embodiments, a light source may be configured to provide a light. A fiber optic light guide operably may be connected to the light source, and transmit the light into the examination space of the medical device.

In some embodiments, the fiber optic light guide and the optical fiber bundle may be disposed coaxially.

In some embodiments, the fiber optic light guide may be disposed around the optical fiber bundle.

In some embodiments, the light may be a structured light having a predefined pattern.

In some embodiments, the system may further include a storage device storing a set of instructions and at least one processor in communication with the storage device. When executing the instructions, the at least one processor is configured to cause the system to obtain a plurality of monitoring images at different moments, determine a reference image associated with the structured light and determine a motion of the subject based on the plurality of monitoring images and the reference image.

In some embodiments, the at least one processor may be further configured to cause the system to obtain scanning data of the ROI of the subject based on the motion of the subject, and reconstruct a scanning image based on the scanning data.

In some embodiments, a guide member may be configured to facilitate a position adjustment of the optical assembly.

In some embodiments, a motion determination module may be configured to determine a motion of the subject based on the monitoring image of the ROI.

In some embodiments, the optical assembly may include one or more lenses.

In a second aspect of the present disclosure, a method for monitoring a subject positioned in a medical device may be provided. The method may include obtaining, via an optical assembly, an optical signal associated with a region of interest (ROI) of the subject in an examination space of the medical device. The method may include transmitting, via an optical fiber bundle, the optical signal to an optical sensing device. The method may include converting, via the optical sensing device, the optical signal to an electrical signal. The method may further include generating, via an image generator, a monitoring image of the ROI based on the electrical signal.

In some embodiments, the method may further include generating, via a light source, a light. The method may further include projecting, via a fiber optic light guide, the light on the ROI of the subject.

In some embodiments, the light may be a structured light having a predefined pattern.

In some embodiments, the method may further include obtaining a plurality of monitoring images at different moments, determining a reference image associated with the structured light, and determining a motion of the subject based on the plurality of monitoring images and the reference image.

In some embodiments, the method may further include obtaining scanning data of the ROI of the subject based on the motion of the subject, and reconstructing a scanning image based on the scanning data.

In some embodiments, the method may further include transmitting, via a first beam splitter, the optical signal from the optical fiber bundle to the optical sensing device, converting, via a projector, a second electrical signal relating to a second image to a second optical signal; transmitting, via the projector, the second optical signal to the first beam splitter; and transmitting, via the first beam splitter, the second optical signal to the optical fiber bundle.

In some embodiments, the method may further include transmitting, via a second beam splitter, the optical signal from the optical assembly to the optical fiber bundle; and transmitting, via the second beam splitter, the second optical signal from the optical fiber bundle to a display region such that the second image relating to the second optical signal is presented on the display region.

In a third aspect of the present disclosure, a system associated with an imaging device may be provided. The system may include a storage device storing a set of instructions and at least one processor in communication with the storage device. When executing the instructions, the at least one processor is configured to cause the imaging system to perform one or more operations as follows. The at least one processor may cause an optical sensing device to convert an optical signal associated with a region of interest (ROI) of a subject in an examination space of the medical device to an electrical signal. The at least one processor may cause an image generator to generate, based on the electrical signal, a monitoring image of the ROI. The optical signal may be obtained according to a process. The process may include obtaining, via an optical assembly, the optical signal associated with the ROI; and transmitting, via an optical fiber bundle, the optical signal to the optical sensing device.

Additional features will be set forth in part in the description which follows, and in part will become apparent to those skilled in the art upon examination of the following and the accompanying drawings or may be learned by production or operation of the examples. The features of the present disclosure may be realized and attained by practice or use of various aspects of the methodologies, instrumentalities, and combinations set forth in the detailed examples discussed below.

### BRIEF DESCRIPTION OF THE DRAWINGS

The present disclosure is further described in terms of exemplary embodiments. These exemplary embodiments are described in detail with reference to the drawings. These embodiments are non-limiting exemplary embodiments, in which like reference numerals represent similar structures throughout the several views of the drawings, and wherein:

FIG. 1 is a schematic diagram illustrating an exemplary imaging system according to some embodiments of the present disclosure;

FIG. 2 is a schematic diagram illustrating exemplary hardware and/or software components of a computing device on which a processing device may be implemented according to some embodiments of the present disclosure;

FIG. 3 is a schematic diagram illustrating exemplary hardware and/or software components of a mobile device on which a terminal may be implemented according to some embodiments of the present disclosure;

FIGS. 4A-4C are schematic diagrams illustrating exemplary monitoring systems for a medical device according to some embodiments of the present disclosure;

FIG. 4D is a schematic diagram illustrating exemplary patterns of a structured light according to some embodiments of the present disclosure;

FIG. 5 is a block diagram illustrating an exemplary processing device according to some embodiments of the present disclosure;

FIG. 6 is a flowchart illustrating an exemplary monitoring process according to some embodiments of the present disclosure;

FIG. 7 is a flowchart illustrating an exemplary process for reconstructing a scanning image of a subject according to some embodiments of the present disclosure; and

FIG. 8 is a flowchart illustrating an exemplary process for reconstructing a scanning image of a subject according to some embodiments of the present disclosure.

### DETAILED DESCRIPTION

In the following detailed description, numerous specific details are set forth by way of examples in order to provide a thorough understanding of the relevant disclosure. However, it should be apparent to those skilled in the art that the present disclosure may be practiced without such details. In other instances, well-known methods, procedures, systems, components, and/or circuitry have been described at a relatively high-level, without detail, in order to avoid unnecessarily obscuring aspects of the present disclosure. Various modifications to the disclosed embodiments will be readily apparent to those skilled in the art, and the general principles defined herein may be applied to other embodiments and applications without departing from the spirit and scope of the present disclosure. Thus, the present disclosure is not limited to the embodiments shown, but to be accorded the widest scope consistent with the claims.

The terminology used herein is for the purpose of describing particular example embodiments only and is not intended to be limiting. As used herein, the singular forms “a,” “an,” and “the” may be intended to include the plural forms as well, unless the context clearly indicates otherwise. It will be further understood that the terms “comprise,” “comprises,” and/or “comprising,” “include,” “includes,” and/or “including,” when used in this specification, specify the presence of stated features, integers, steps, operations, elements, and/or components, but do not preclude the presence or addition of one or more other features, integers, steps, operations, elements, components, and/or groups thereof.

It will be understood that the term “system,” “engine,” “unit,” “module,” and/or “block” used herein are one method to distinguish different components, elements, parts, section or assembly of different level in ascending order. However, the terms may be displaced by another expression if they may achieve the same purpose.

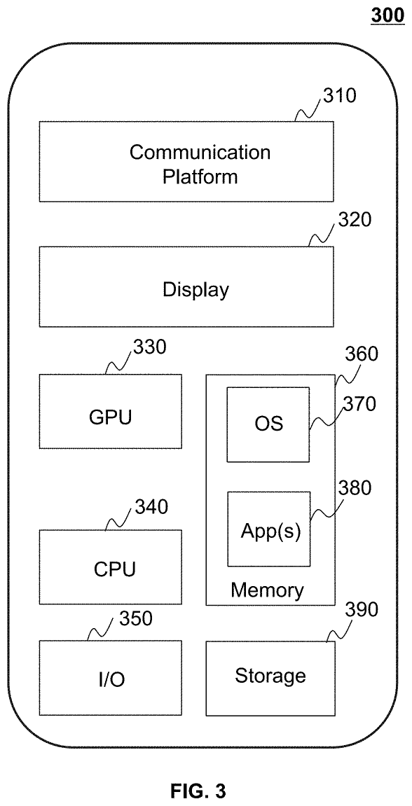

Generally, the word “module,” “unit,” or “block,” as used herein, refers to logic embodied in hardware or firmware, or to a collection of software instructions. A module, a unit, or a block described herein may be implemented as software and/or hardware and may be stored in any type of non-transitory computer-readable medium or another storage device. In some embodiments, a software module/unit/block may be compiled and linked into an executable program. It will be appreciated that software modules can be callable from other modules/units/blocks or from themselves, and/or may be invoked in response to detected events or interrupts. Software modules/units/blocks configured for execution on computing devices (e.g., processor 310 as illustrated in FIG. 3) may be provided on a computer-readable medium, such as a compact disc, a digital video disc, a flash drive, a magnetic disc, or any other tangible medium, or as a digital download (and can be originally stored in a compressed or installable format that needs installation, decompression, or decryption prior to execution). Such software code may be stored, partially or fully, on a storage device of the executing computing device, for execution by the computing device. Software instructions may be embedded in firmware, such as an Erasable Programmable Read Only Memory (EPROM). It will be further appreciated that hardware modules/units/blocks may be included in connected logic components, such as gates and flip-flops, and/or can be included of programmable units, such as programmable gate arrays or processors. The modules/units/blocks or computing device functionality described herein may be implemented as software modules/units/blocks, but may be represented in hardware or firmware. In general, the modules/units/blocks described herein refer to logical modules/units/blocks that may be combined with other modules/units/blocks or divided into sub-modules/sub-units/sub-blocks despite their physical organization or storage. The description may be applicable to a system, an engine, or a portion thereof.

It will be understood that when a unit, engine, module or block is referred to as being “on,” “connected to,” or “coupled to,” another unit, engine, module, or block, it may be directly on, connected or coupled to, or communicate with the other unit, engine, module, or block, or an intervening unit, engine, module, or block may be present, unless the context clearly indicates otherwise. As used herein, the term “and/or” includes any and all combinations of one or more of the associated listed items.

These and other features, and characteristics of the present disclosure, as well as the methods of operation and functions of the related elements of structure and the combination of parts and economies of manufacture, may become more apparent upon consideration of the following description with reference to the accompanying drawings, all of which form a part of this disclosure. It is to be expressly understood, however, that the drawings are for the purpose of illustration and description only and are not intended to limit the scope of the present disclosure. It is understood that the drawings are not to scale.

The following description is provided with reference to exemplary embodiments that the medical device includes a scanning device (e.g., an MRI scanner) unless otherwise stated. However, it is understood that it is for illustration purposes only and not intended to limit the scope of the present disclosure. The system and method disclosed herein may be suitable for other applications. Merely by way of example, the medical device may include a radiotherapy device (e.g., an image-guided radiotherapy (IGRT) device); and/or the systems and methods for monitoring the status of the subject may be configured for the radiotherapy device.

Various medical devices can be widely used to scan a subject (e.g., a patient) and conduct a primary diagnosis so that doctors can better understand the patient's condition, and develop a corresponding treatment plan accordingly. Common medical devices may include a scanning bore, such as a magnetic resonance (MR) scanner, a computed tomography (CT) scanner, and so on. When using such medical device with the scanning bore to scan the patient, it is necessary to monitor the patient's status (e.g., a motion) to avoid problems (such as a poor scan quality caused by the patient movement), and monitor whether an emergency occurs (such as vomiting or suffocation).

In some conventional cases, a commercial camera is directly installed in the scanning bore of the medical device for real-time monitoring of the patient's status. However, because an electric connection of the camera device is implemented through many cables, cable wrapping can be introduced. The installation of the camera and the cables may be relatively complicated. A limited examination space of the scanning bore may be occupied by the camera and/or the cables. Moreover, for an MR scanner with high electromagnetic compatibility requirements, such camera may generate signal interference during operation, which may affect the MR scanner operate normally. Moreover, the camera generally includes metallic materials (e.g., a metal casing), which can also introduce signal interference. A monitoring system having a good compatibility with the medical device is desirable.

Various embodiments of the present disclosure may be provided as a monitoring system configured to monitor the subject in real time or near real time during the medical device operates. In some embodiments, the monitoring system may include an optical assembly, an optical fiber bundle, an optical sensing device and an image generator. The optical assembly may acquire an optical signal reflected or scattered by a region of interest (ROI) of a subject in an examination space of the medical device. The optical fiber bundle may receive the optical signal and transmit the optical signal to the optical sensing device. The optical sensing device may convert the optical signal to an electrical signal. The image generator may generate a monitoring image based on the electrical signal. The monitoring image may be displayed on a display device. According to the monitoring image, the status of the subject may be monitored in real time or near real time. For the monitoring system, only one optical fiber bundle may be arranged in the medical device, and may be optically coupled to the optical assembly to transmit optical signal(s) from the optical assembly. It is unnecessary to arrange addition cables for the optical assembly, and the installation of the monitoring system can be facilitated. The optical fiber bundle may not occupy much of the limited examination space of the medical device, and the monitoring to the subject can be facilitated. Besides, the optical assembly and the optical fiber bundle are made of non-metallic materials, which may not bring image artifacts to the monitoring image. The image quality of the monitoring image may be improved.

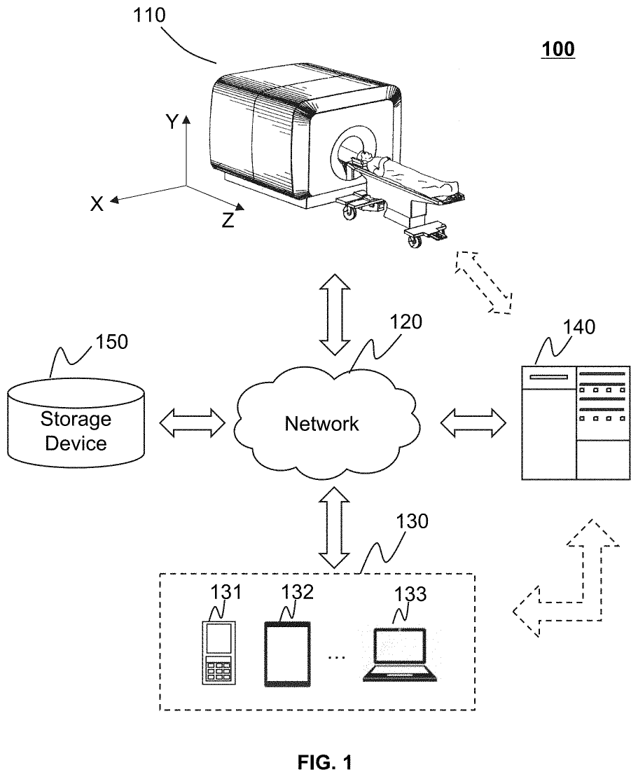

FIG. 1 is a schematic diagram illustrating an exemplary imaging system according to some embodiments of the present disclosure. The imaging system 100 may include a computed tomography (CT) system, a computed tomography angiography (CTA) system, a positron emission tomography (PET) system, a single photon emission computed tomography (SPECT) system, a magnetic resonance imaging (MRI) system, or the like, or a combination thereof. In some embodiments, the imaging system 100 may be a single modality system (e.g., the CT system, the MRI system). In some embodiments, the imaging system may be a multi-modality system (e.g., a PET-CT system, a PET-MRI system).

As illustrated in FIG. 1, the imaging system 100 may include a scanning device 110, a network 120, one or more terminals 130, a processing device 140, and a storage device 150. The components of the imaging system 100 may be connected in one or more of various ways. Mere by way of example, as illustrated in FIG. 1, the scanning device 110 may be connected to the processing device 140 through the network 120. As another example, the scanning device 110 may be connected to the processing device 140 directly (as indicated by the bi-directional arrow in dotted lines linking the scanning device 110 and the processing device 140). As a further example, the storage device 150 may be connected to the processing device 140 directly or through the network 120. As still a further example, a terminal device (e.g., a mobile device 131, a tablet computer 132, a laptop computer 133, etc.) may be connected to the processing device 140 directly (as indicated by the bi-directional arrow in dotted lines linking the terminal 130 and the processing device 140) or through the network 120.

Taking an MRI scanner as an example, the scanning device 110 may scan an object located within its examination space and generate a plurality of data relating to the object. In the present disclosure, “subject” and “object” are used interchangeably. Mere by way of example, the object may include a patient, a man-made object, etc. As another example, the object may include a specific portion, organ, and/or tissue of a patient. For example, the object may include a head, a brain, a neck, a body, a shoulder, an arm, a thorax, a heart, a stomach, a blood vessel, a soft tissue, a knee, feet, or the like, or any combination thereof. In some embodiments, the scanning device 110 may be a close-bore scanner or an open-bore scanner. The scanning device 110 may include a magnet assembly, a gradient coil assembly, a radiofrequency (RF) coil assembly and a scanning table.

The magnet assembly may generate a main magnetic field for polarizing the subject to be scanned. For example, the magnet assembly may include a permanent magnet, a superconducting electromagnet, a resistive electromagnet, etc.

The gradient coil assembly may generate a gradient magnetic field. The gradient coil assembly may generate one or more magnetic field gradient pulses to the main magnetic field in the X direction (Gx), Y direction (Gy), and Z direction (Gz) to encode the spatial information of the subject. As shown in FIG. 1, the X axis, the Y axis, and the Z axis shown in FIG. 1 may form an orthogonal coordinate system. The X axis and the Z axis shown in FIG. 1 may be horizontal, and the Y axis may be vertical. As illustrated, the positive X direction along the X axis may be from the right side to the left side of the scanning device 110 seen from the direction facing the front of the scanning device 110; the positive Y direction along the Y axis shown in FIG. 1 may be from the lower part to the upper part of the scanning device 110; the positive Z direction along the Z axis shown in FIG. 1 may refer to a direction in which the object is moved out of a scanning bore of the scanning device 110. The examination space may be in the scanning bore. The examination space may be a portion of the scanning bore.

The RF coil assembly may include a plurality of RF coils. The plurality of RF coils may include one or more RF transmitting coils and/or one or more RF receiving coils. The RF transmitting coil(s) may transmit RF pulses to the subject. Under the coordinated action of the main magnetic field, the gradient magnetic field, and the RF pulses, MR signals relating to the subject may be generated. The RF receiving coils may receive MR signals from the subject. In some embodiments, one or more RF coils may both transmit RF pulses and receive MR signals at different times. In some embodiments, the function, size, type, geometry, position, amount, and/or magnitude of the RF coil(s) may be determined or changed according to one or more specific conditions. For example, according to the difference in function and size, the RF coil(s) may be classified as volume coils and local coils. The term “volume coil” as used herein generally refers to coils that are used to provide a homogenous RF excitation field across a relatively large volume, such as to cover the entire body of the subject. For example, many commercially available MRI scanners may include a volume coil that is big enough for whole body imaging of a human subject, and thus sometimes the volume coil may be referred to as a “body coil”. The term “local coil” as used herein may refer to coils that are to be placed in close proximity to a region of interest of the subject during MR imaging. The local coils may be designed to achieve improved RF detection sensitivity over a small region of interest (ROI). In some embodiments, an RF receiver coil may correspond to a channel. The RF receiving coil(s) may receive a plurality of channels of MR signals from the subject. The received MR signal(s) may be sent to the processing device 140 directly or via the network 120 for image reconstruction and/or image processing.

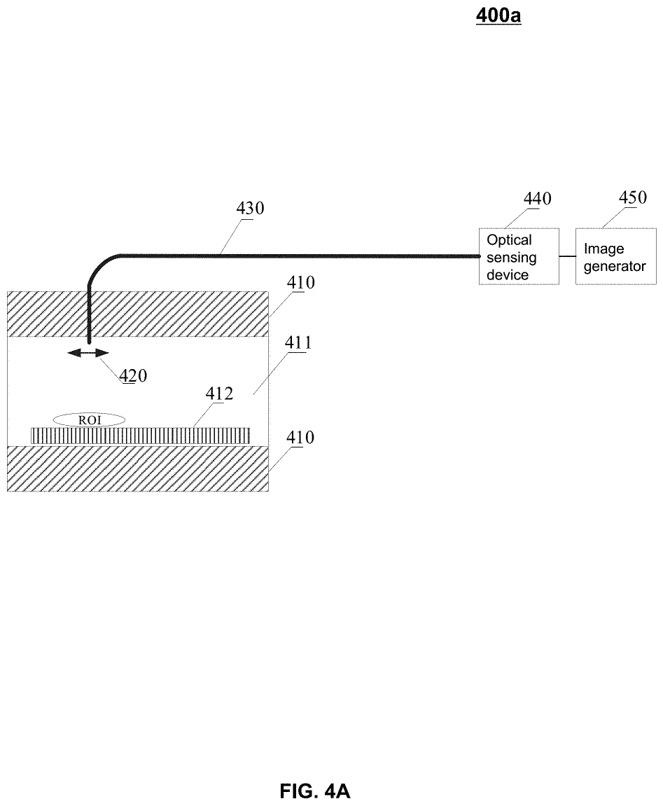

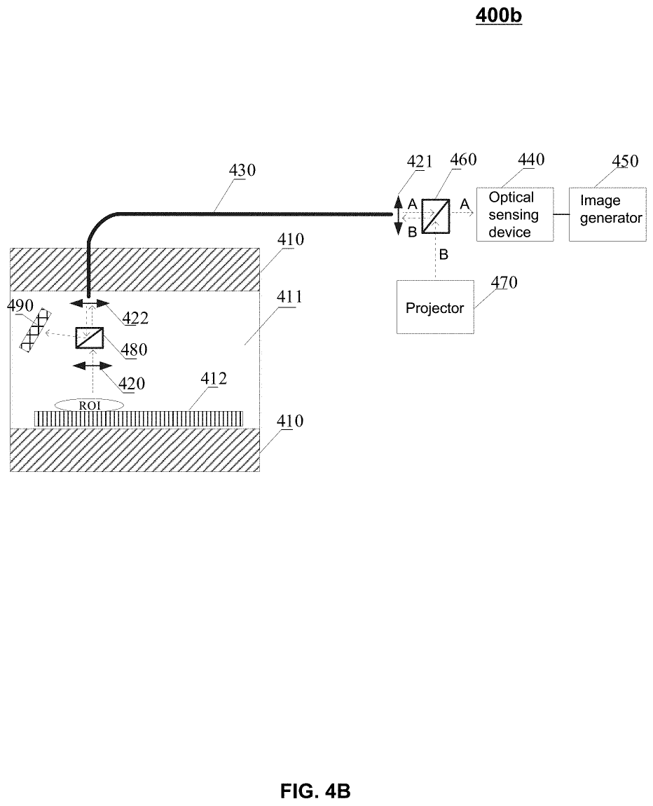

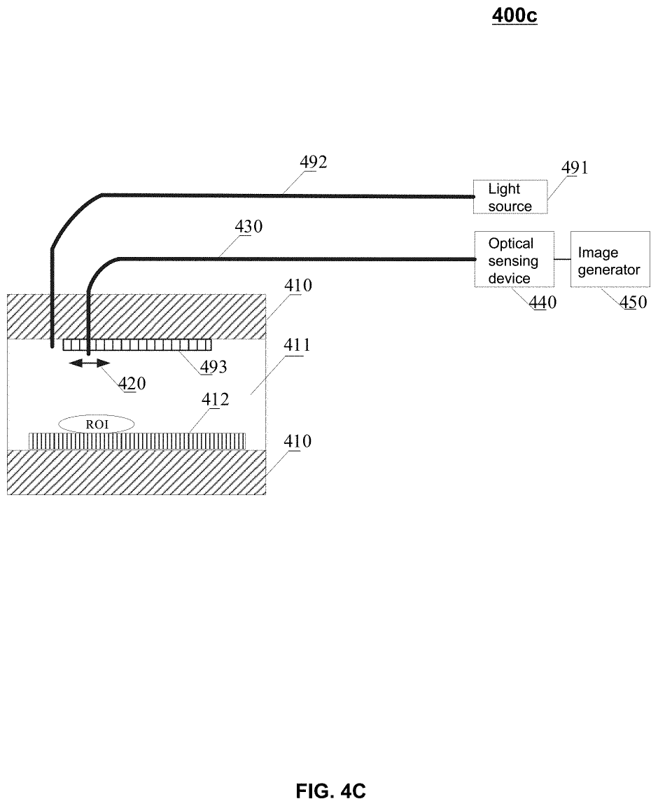

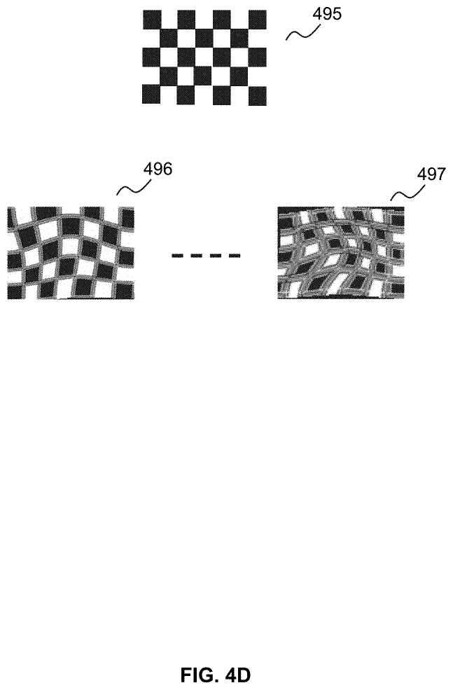

In some embodiments, the imaging system 100 may further include a monitoring system (not shown in FIG. 1) configured to generate one or more images of the subject (also referred to as monitoring image(s)) and/or monitor a status of the subject. In some embodiments, at least a portion of the monitoring system (as shown in FIGS. 4A-4C) may be operably coupled to or integrated in the scanning device 110. In some embodiments, at least a portion of the monitoring system may be part of the scanning device 110. One or more components of the monitoring system may be disposed in the scanning device 110. For example, an optical assembly 420 (shown in FIG. 4A) of the monitoring system may be disposed in the scanning bore 411 of the scanning device 410 (same as or similar to the scanning device 110), and configured to acquire optical signal related to the subject. In some embodiments, the monitoring system may be part of the imaging system 100. In some embodiments, the monitoring system may be separated from (or independent of) the imaging system 100 in functionality. The imaging system 100 may obtain data/information relating to the subject from the monitoring system. More descriptions regarding the monitoring system may be found elsewhere in the present disclosure (e.g., FIGS. 4A-4C, and the descriptions thereof).

The network 120 may include any suitable network that can facilitate the exchange of information and/or data for the imaging system 100. In some embodiments, one or more components of the imaging system 100 (e.g., the scanning device 110, the terminal 130, the processing device 140, the storage device 150, or one or more components of the monitoring system) may communicate information and/or data with one or more other components of the imaging system 100 via the network 120. For example, the processing device 140 may obtain scanning data from the scanning device 110 via the network 120. As another example, the processing device 140 may obtain a monitoring image from the monitoring system via the network 120. As a further example, the processing device 140 may obtain signals from the monitoring system via the network 120 and generate monitoring image(s). In some embodiments, the network 120 may be any type of wired or wireless network, or a combination thereof. The network 120 may be and/or include a public network (e.g., the Internet), a private network (e.g., a local area network (LAN), a wide area network (WAN)), etc.), a wired network (e.g., an Ethernet network), a wireless network (e.g., an 802.11 network, a Wi-Fi network, etc.), a cellular network (e.g., a Long Term Evolution (LTE) network), a frame relay network, a virtual private network (“VPN”), a satellite network, a telephone network, routers, hubs, switches, server computers, and/or any combination thereof. Merely by way of example, the network 120 may include a cable network, a wireline network, a fiber-optic network, a telecommunications network, an intranet, a wireless local area network (WLAN), a metropolitan area network (MAN), a public telephone switched network (PSTN), a Bluetooth™ network, a ZigBee™ network, a near field communication (NFC) network, or the like, or any combination thereof. In some embodiments, the network 120 may include one or more network access points. For example, the network 120 may include wired and/or wireless network access points such as base stations and/or internet exchange points through which one or more components of the imaging system 100 may be connected to the network 120 to exchange data and/or information.

The terminal 130 may include a mobile device 131, a tablet computer 132, a laptop computer 133, or the like, or any combination thereof. In some embodiments, the mobile device 131 may include a smart home device, a wearable device, a smart mobile device, a virtual reality device, an augmented reality device, or the like, or any combination thereof. In some embodiments, the smart home device may include a smart lighting device, a control device of an intelligent electrical apparatus, a smart monitoring device, a smart television, a smart video camera, an interphone, or the like, or any combination thereof. In some embodiments, the wearable device may include a smart bracelet, smart footgear, a pair of smart glasses, a smart helmet, a smart watch, smart clothing, a smart backpack, a smart accessory, or the like, or any combination thereof. In some embodiments, the smart mobile device may include a smartphone, a personal digital assistant (PDA), a gaming device, a navigation device, a point of sale (POS) device, or the like, or any combination thereof. In some embodiments, the virtual reality device and/or the augmented reality device may include a virtual reality helmet, a virtual reality glass, a virtual reality patch, an augmented reality helmet, an augmented reality glass, an augmented reality patch, or the like, or any combination thereof. For example, the virtual reality device and/or the augmented reality device may include a Google™ Glass, an Oculus Rift, a Hololens, a Gear VR, etc. In some embodiments, the terminal 130 may remotely operate the scanning device 110 and/or the processing device 140. For example, the terminal 130 may remotely operate the processing device 140 to cause the monitoring system to monitor the subject during the scan. In some embodiments, the terminal 130 may operate the scanning device 110 and/or the processing device 140 via a wireless connection. In some embodiments, the terminal 130 may receive information and/or instructions inputted by a user, and send the received information and/or instructions to the scanning device 110 or to the processing device 140 via the network 120. In some embodiments, the terminal 130 may receive data and/or information from the processing device 140. In some embodiments, the terminal 130 may be part of the processing device 140. In some embodiments, the terminal 130 may be omitted.

The processing device 140 may process data and/or information obtained from the scanning device 110, the terminal 130, the monitoring system, and/or the storage device 150. For example, the processing device 140 may obtain scanning data from the scanning device 110, and reconstruct a scanning image based on the scanning data. As another example, the processing device 140 may obtain a plurality of monitoring images generated at different time points, and determine a motion of the subject based on the plurality of monitoring images. As a further example, the processing device 140 may obtain signals or information from the monitoring system and generate one or more monitoring images.

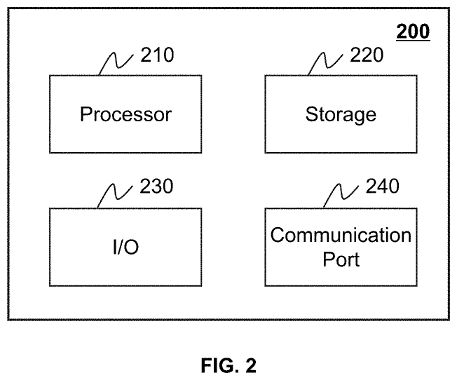

In some embodiments, the processing device 140 may be a single server, or a server group. The server group may be centralized or distributed. In some embodiments, the processing device 140 may be local or remote. For example, the processing device 140 may access information and/or data stored in or acquired by the scanning device 110, the terminal 130, the monitoring system, and/or the storage device 150 via the network 120. As another example, the processing device 140 may be directly connected to the scanning device 110 (as illustrated by the bidirectional arrow in dashed lines connecting the processing device 140 and the scanning device 110 in FIG. 1), the terminal 130 (as illustrated by the bidirectional arrow in dashed lines connecting the processing device 140 and the terminal 130 in FIG. 1), and/or the storage device 150 to access stored or acquired information and/or data. In some embodiments, the processing device 140 may be implemented on a cloud platform. Merely by way of example, the cloud platform may include a private cloud, a public cloud, a hybrid cloud, a community cloud, a distributed cloud, an inter-cloud, a multi-cloud, or the like, or any combination thereof. In some embodiments, the processing device 140 may be implemented on a computing device 200 having one or more components illustrated in FIG. 2 or a mobile device 300 having one or more components illustrated in FIG. 3 in the present disclosure.

The storage device 150 may store data and/or instructions. In some embodiments, the storage device 150 may store data obtained from the scanning device 110, the terminal 130, the monitoring system, and/or the processing device 140. In some embodiments, the storage device 150 may store data and/or instructions that the processing device 140 may execute or use to perform exemplary methods described in the present disclosure. In some embodiments, the storage device 150 may include a mass storage device, a removable storage device, a volatile read-and-write memory, a read-only memory (ROM), or the like, or any combination thereof. Exemplary mass storage devices may include a magnetic disk, an optical disk, a solid-state drive, etc. Exemplary removable storage devices may include a flash drive, a floppy disk, an optical disk, a memory card, a zip disk, a magnetic tape, etc. Exemplary volatile read-and-write memory may include a random access memory (RAM). Exemplary RAM may include a dynamic RAM (DRAM), a double date rate synchronous dynamic RAM (DDR SDRAM), a static RAM (SRAM), a thyristor RAM (T-RAM), and a zero-capacitor RAM (Z-RAM), etc. Exemplary ROM may include a mask ROM (MROM), a programmable ROM (PROM), an erasable programmable ROM (PEROM), an electrically erasable programmable ROM (EEPROM), a compact disk ROM (CD-ROM), and a digital versatile disk ROM, etc. In some embodiments, the storage device 150 may be implemented on a cloud platform. Merely by way of example, the cloud platform may include a private cloud, a public cloud, a hybrid cloud, a community cloud, a distributed cloud, an inter-cloud, a multi-cloud, or the like, or any combination thereof.

In some embodiments, the storage device 150 may be connected to the network 120 to communicate with one or more components of the imaging system 100 (e.g., the scanning device 110, the processing device 140, the terminal 130, the monitoring system, etc.). One or more components of the imaging system 100 may access the data or instructions stored in the storage device 150 via the network 120. In some embodiments, the storage device 150 may be directly connected to or communicate with one or more components of the imaging system 100 (e.g., the scanning device 110, the processing device 140, the terminal 130, the monitoring system, etc.). In some embodiments, the storage device 150 may be part of the processing device 140.

In some embodiments, the imaging system 100 may further include one or more power supplies (not shown in FIG. 1) connected to one or more components of the imaging system 100 (e.g., the scanning device 110, the processing device 140, the terminal 130, the storage device 150, the monitoring system, etc.). It should be noted that the imaging system 100 and the scanning device 110 illustrated in FIG. 1 are merely provided for the purposes of illustration, and not intended to limit the scope of the present disclosure. For persons having ordinary skills in the art, multiple variations and modifications may be made under the teachings of the present disclosure. However, those variations and modifications do not depart from the scope of the present disclosure. For example, the imaging system 100 may be replaced by a treatment system, and accordingly, the scanning device 110 may be replaced by a treatment device (e.g., a radiotherapy device). That is, the monitoring system may be alternatively coupled to the treatment system, and configured to monitor the status of the subject before, during, and/or after a treatment process.

FIG. 2 is a schematic diagram illustrating exemplary hardware and/or software components of a computing device on which a processing device may be implemented according to some embodiments of the present disclosure. As illustrated in FIG. 2, computing device 200 may include a processor 210, a storage 220, an input/output (I/O) 230, and a communication port 240.

The processor 210 may execute computer instructions (e.g., program code) and perform functions of the processing device 140 in accordance with techniques described herein. The computer instructions may include routines, programs, objects, components, signals, data structures, procedures, modules, and functions, which perform particular functions described herein. For example, the processor 210 may process data obtained from the scanning device 110, the terminal 130, the storage device 150, the monitoring system, and/or any other component of the imaging system 100. In some embodiments, the processor 210 may include a microcontroller, a microprocessor, a reduced instruction set computer (RISC), an application specific integrated circuits (ASICs), an application-specific instruction-set processor (ASIP), a central processing unit (CPU), a graphics processing unit (GPU), a physics processing unit (PPU), a microcontroller unit, a digital signal processor (DSP), a field programmable gate array (FPGA), an advanced RISC machine (ARM), a programmable logic device (PLD), any circuit or processor capable of executing one or more functions, or the like, or any combinations thereof.

Merely for illustration purposes, only one processor is described in the computing device 200. However, it should be noted that the computing device 200 in the present disclosure may also include multiple processors, and thus operations of a method that are performed by one processor as described in the present disclosure may also be jointly or separately performed by the multiple processors. For example, if in the present disclosure the processor of the computing device 200 executes both operations A and B, it should be understood that operations A and step B may also be performed by two different processors jointly or separately in the computing device 200 (e.g., a first processor executes operation A and a second processor executes operation B, or the first and second processors jointly execute operations A and B).

The storage 220 may store data/information obtained from the scanning device 110, the terminal 130, the storage device 150, the monitoring system or any other component of the imaging system 100. In some embodiments, the storage 220 may include a mass storage device, a removable storage device, a volatile read-and-write memory, a read-only memory (ROM), or the like, or any combination thereof. For example, the mass storage device may include a magnetic disk, an optical disk, a solid-state drive, etc. The removable storage device may include a flash drive, a floppy disk, an optical disk, a memory card, a zip disk, a magnetic tape, etc. The volatile read-and-write memory may include a random access memory (RAM). The RAM may include a dynamic RAM (DRAM), a double date rate synchronous dynamic RAM (DDR SDRAM), a static RAM (SRAM), a thyristor RAM (T-RAM), and a zero-capacitor RAM (Z-RAM), etc. The ROM may include a mask ROM (MROM), a programmable ROM (PROM), an erasable programmable ROM (PEROM), an electrically erasable programmable ROM (EEPROM), a compact disk ROM (CD-ROM), and a digital versatile disk ROM, etc. In some embodiments, the storage 220 may store one or more programs and/or instructions to perform exemplary methods described in the present disclosure.

The I/O 230 may input or output signals, data, or information. In some embodiments, the I/O 230 may enable user interaction with the processing device 140. In some embodiments, the I/O 230 may include an input device and an output device. Exemplary input devices may include a keyboard, a mouse, a touch screen, a microphone, a trackball, or the like, or a combination thereof. Exemplary output devices may include a display device, a loudspeaker, a printer, a projector, or the like, or a combination thereof. Exemplary display devices may include a liquid crystal display (LCD), a light-emitting diode (LED)-based display, a flat panel display, a curved screen, a television device, a cathode ray tube (CRT), or the like, or a combination thereof.

Merely by way of example, a user (e.g., an operator) may input data related to an object (e.g., a patient) that is being/to be imaged/scanned (or treated) through the I/O 230. The data related to the object may include identification information (e.g., the name, age, gender, medical history, contact information, physical examination result, etc.) and/or the test information including the nature of the scan that must be performed. The user may also input parameters needed for the operation of the scanning device 110, such as image contrast and/or ratio, a region of interest (ROI), slice thickness, an imaging type (e.g., T1 weighted imaging, T2 weighted imaging, proton density weighted imaging, etc.), T1, T2, an echo type (spin echo, fast spin echo (FSE), fast recovery FSE, single shot FSE, gradient recalled echo, fast imaging with steady-state procession, and so on), a flip angle value, acquisition time (TA), echo time (TE), repetition time (TR), echo train length (ETL), the number of phases, the number of excitations (NEX), inversion time, bandwidth (e.g., RF receiver bandwidth, RF transmitter bandwidth, etc.), a scan type, a type of sampling, or the like, or any combination thereof. The I/O 230 may also display images generated based on sampled data.

The communication port 240 may be connected to a network (e.g., the network 120) to facilitate data communications. The communication port 240 may establish connections between the processing device 140 and the scanning device 110, the terminal 130, the storage device 150, or one or more components of the monitoring system. The connection may be a wired connection, a wireless connection, or a combination of both that enables data transmission and reception. The wired connection may include an electrical cable, an optical cable, a telephone wire, or the like, or any combination thereof. The wireless connection may include Bluetooth, Wi-Fi, WiMax, WLAN, ZigBee, mobile network (e.g., 3G, 4G, 5G, etc.), or the like, or a combination thereof. In some embodiments, the communication port 240 may be a standardized communication port, such as RS232, RS485, etc. In some embodiments, the communication port 240 may be a specially designed communication port. For example, the communication port 240 may be designed in accordance with the digital imaging and communications in medicine (DICOM) protocol.

FIG. 3 is a schematic diagram illustrating exemplary hardware and/or software components of a mobile device on which a terminal may be implemented according to some embodiments of the present disclosure. As illustrated in FIG. 3, the mobile device 300 may include a communication platform 310, a display 320, a graphic processing unit (GPU) 330, a central processing unit (CPU) 340, an I/O 350, a memory 360, and a storage 390. In some embodiments, any other suitable component, including but not limited to a system bus or a controller (not shown), may also be included in the mobile device 300. In some embodiments, a mobile operating system 370 (e.g., iOS, Android, Windows Phone, Harmony OS, etc.) and one or more applications 380 may be loaded into the memory 360 from the storage 390 in order to be executed by the CPU 340. The applications 380 may include a browser or any other suitable mobile apps for receiving and rendering information relating to image processing or other information from the processing device 140. User interactions with the information stream may be achieved via the I/O 350 and provided to the processing device 140 and/or other components of the imaging system 100 via the network 120.

To implement various modules, units, and their functionalities described in the present disclosure, computer hardware platforms may be used as the hardware platform(s) for one or more of the elements described herein. The hardware elements, operating systems and programming languages of such computers are conventional in nature, and it is presumed that those skilled in the art are adequately familiar therewith to adapt those technologies to generate and track shapes of a target as described herein. A computer with user interface elements may be used to implement a personal computer (PC) or another type of work station or terminal device, although a computer may also act as a server if appropriately programmed. It is believed that those skilled in the art are familiar with the structure, programming and general operation of such computer equipment and as a result the drawings should be self-explanatory.

FIG. 4A is a schematic diagram illustrating an exemplary monitoring system for a medical device according to some embodiments of the present disclosure. The monitoring system may be compatible for the medical device (e.g., a scanning device, a treatment device) in structures and/or functionalities. It should be noted that the following description is provided with reference to exemplary embodiments that the medical device includes a scanning device (e.g., an MRI scanner) unless otherwise stated. A monitoring system used with a treatment device may be similar to the monitoring system used with the scanning device, and relevant descriptions are not repeated herein. The monitoring system 400a illustrated in FIG. 4A may be implemented on the imaging system 100. In some embodiments, the monitoring system 400a may be part of the imaging system 100. In some embodiments, the monitoring system 400a may be provided as an add-on to any medical device, providing medical device manufacturer(s) and/or users the flexibility to conveniently adopt the systems and methods as described in the present disclosure without significant changes to the design or configurations of the medical device.

The monitoring system 400a may be configured to monitor a status of a subject placed in an examination space of the scanning device 110 (e.g., a scanning bore of the scanning device 110). For example, the monitoring system 400a may generate and/or provide one or more monitoring images of at least a portion of the subject (e.g., a region of interest (ROI) of the subject). The status of the subject (e.g., whether the subject feels uncomfortable, or whether the subject moves or not) may be determined based on the one or more monitoring images. In some embodiments, the monitoring system 400a may monitor the status of the subject during a scanning process or treatment process of the scanning device 110, so that the imaging system 100 may instruct and/or adjust the scanning process or treatment process of the scanning device 110 to achieve a relatively high-quality imaging or treatment. In some embodiments, if it is found that the subject feels uncomfortable during a scan of the scanning device 110, a current scan procedure may be suspended and/or adjusted so as to relieve the subject's uncomfortable feeling. For example, if the subject is found nervous, painful, or scared, by analyzing or observing monitoring image(s) generated at a moment or in a time period, the processing device 140 or an operator of the scanning device 110 may take operations (e.g., playing a video on a display region within the subject' view, playing a music, etc.) to relieve the subject's feeling of nervous, painful, or scared. In some embodiments, if it is found that the subject moves during the scan, the imaging system 100 may perform one or more operations to remove or reduce motion artifacts from scanning image(s) generated by the scanning device 110, and thus the quality of the scanning image(s) may be improved. In some embodiments, the monitoring image(s) may include motion related data (or information) of the subject. In some embodiments, the motion related data may be obtained based on a plurality of monitoring images generated at different moments. In some embodiments, the motion artifacts may be removed based on the motion related data.

As illustrated in FIG. 4A, the monitoring system 400a may include an optical assembly 420, an optical fiber bundle 430, an optical sensing device 440, and an image generator 450. In some embodiments, at least a portion of the monitoring system 400a (e.g., the optical assembly 420, a portion of the optical fiber bundle 430) may be operably coupled to or integrated in a scanning device 410.

The scanning device 410 may be the same as or similar to the scanning device 110 illustrated in FIG. 1. For example, the scanning device 410 may be an MRI scanner. The scanning device 410 may be configured to acquire scanning data by scanning the ROI of the subject. The ROI of the subject may include an organ (e.g., a lung, the liver, the stomach), a body part (e.g., the chest, the abdomen, the head), an injured part, a tumor, or the like, or any combination thereof. The scanning device 410 may include a scanning bore 411 for accommodating the subject to be scanned. In some embodiments, the subject may be placed on a scanning table 412 and moved into an examination space in the scanning bore 411. The subject may be moved into or out the scanning bore 411 with the scanning table 412. In some embodiments, the scanning device 410 may be a CT scanner, a PET scanner, an MRI scanner, a PET-CT scanner, a PET-MRI scanner, and so on. For example, if the scanning device 410 is a CT scanner, the scanning device 410 may generate CT scanning data. If the scanning device 410 is an MRI device, the scanning device 410 may generate MR scanning data. If the scanning device 410 is a PET-CT scanner, the scanning device 410 may generate PET-CT scanning data. If the scanning device 410 is a PET-MRI device, the scanning device 410 may generate PET-MRI scanning data.

The optical assembly 420 may be configured to acquire an optical signal associated with the ROI of the subject. The optical assembly 420 may be a light reception device. The optical assembly 420 may include one or more arranged optical elements (e.g., one or more lenses). For example, the optical assembly 420 may include an optical lens or an optical lens group. Exemplary optical elements may include a plano-convex lens, a plano-concave lens, a plane mirror, a beam splitter mirror, a reflecting mirror, an aspherical lens, an optical grating, a plane polarizer, a quarter wave plate (QWP), or the like, or any combination thereof. The one or more optical elements may be combined or arranged reasonably to form the optical assembly 420, for example, based on one or more parameters of the one or more optical elements. Exemplary parameters of the one or more optical elements may include a focus length, a numerical aperture (N/A), a field of view (FOV), an operating wavelength, a depth of field, a refractive index, a transmittance, or the like, or any combination thereof. In some embodiments, the parameter(s) may be selected based on requirements of the monitoring system 400a, such as a size of the scanning bore 411, a size of the scanning table 412, a size of the ROI to be monitored. The optical assembly 420 may be designed according to monitoring requirements. For example, the optical lens of different parameters may be adjusted according to the size of the ROI to be monitored. In some embodiments, the optical elements may be made of non-metallic materials including for example, a plastic material, a resin, or a glass material, or the like, or a combination thereof. In some embodiments, the optical assembly 420 may include a non-metallic casing (not shown in FIG. 4A). The one or more optical elements may be housed in the non-metallic casing so that the optical assembly 420 can be mounted easily. Compared with a conventional light reception device (e.g., a camera including metal materials), the optical assembly 420 may be totally made of non-metallic materials, thereby avoiding or reducing signal interferences with the scanning device 410 during the scan. Besides, in some embodiments, the operation of optical assembly 420 don't need to be driven by additional supply circuits. The optical assembly 420 may collect the optical signal automatically upon receipt of the optical signal. The optical assembly 420 may not cause signal interference, which has a good compatibility with the scanning device 410. Regardless of the position of the optical assembly 420 in the scanning device 410, the optical assembly 420 may not cause additional noises into the scanning data (e.g., MR signals), and may not affect the quality of the scanning image(s).

In some embodiments, for different scanning devices, such as an MR scanner, a CT scanner, or a RT scanner, the optical assembly 420 may be specifically designed according to compatibility requirements of these scanning devices. For example, for the MR scanner, the optical assembly 420 may not be made of ferro magnetic materials (e.g., Fe, Co, Ni) in order to reduce or avoid signal interference. As another example, for the CT scanner, the optical assembly 420 may not be made of materials having low transmittance for X-rays, such as metallic materials. As a further example, for the CT/RT scanner, the optical assembly 420 may be made of radiation-resistant materials lest that radioactive rays damage the one or more optical elements of the optical assembly 420.

The optical assembly 420 may be compatible for the scanning device 410. The optical assembly 420 may be located in or be movable in or to an examination space of the scanning device 410. In some embodiments, the position of the optical assembly 420 may be set according to a target ROI (to be monitored) of the subject. In some embodiments, as shown in FIG. 4A, the optical assembly 420 may be mounted on a wall (e.g., an inner wall) of the scanning bore 411. For example, the optical assembly 420 may be embedded into the wall of the scanning bore 411. In some embodiments, a surface (e.g., a top, or a bottom) of the optical assembly 420 (e.g., a surface of a lens of the optical assembly 420) may be kept at a same level as the wall of the scanning bore 411, so that the optical assembly 420 occupies no space or only a relatively small portion of the examination space in the scanning bore 411, and a neatness of the scanning bore 411 is ensured. In some embodiments, the optical assembly 420 may be mounted such that the surface of the optical assembly 420 is a distance away from the wall of the scanning bore 411. For example, the optical assembly 420 may hang in the examination space of the scanning bore 411. In some embodiments, the height of the optical assembly 420 may be adjusted according to a lifting device. For example, the height of the optical assembly 420 may be adjusted by adjusting the lifting device manually or automatically. If it is desired to implement a relatively wide optical signal acquisition range, the optical assembly 420 may be moved to stay away from the ROI of the subject. In some embodiments, the optical assembly 420 may be mounted on the scanning table 412 (not shown in FIG. 4A). The scanning table 412 may carry the optical assembly 420 to move into the examination space of the scanning bore 411.

It should be noted that the position of the optical assembly 420 may be not intended to be limiting. For example, the position of the optical assembly 420 may be set according to the ROI of the subject. The optical assembly 420 may have an optical signal acquisition range. The optical signal acquisition range may be related to a position of the optical assembly 420 with respect to the scanning bore 411, a viewing angle of the optical assembly 420, and the parameters of the one or more optical elements. The optical signal acquisition range of the optical assembly 420 may need to cover the ROI of the subject in order to acquire optical signal(s) reflected or scattered by the ROI of the subject. In some embodiments, the optical signal may be a scattered light and/or a reflected light. Merely for illustration, if the ROI of the subject is the head of a patient, the optical signal acquisition range of the optical assembly 420 may include a space covering the head of the subject. The optical assembly 420 may receive or acquire the optical signal reflected or scattered by the head. The optical signal may be further processed to form a monitoring image showing the head of the subject.

The optical fiber bundle 430 may be configured to transmit optical signal(s). The optical fiber bundle 430 may include a first end and a second end. As shown in FIG. 4A, the first end of the optical fiber bundle 430 may be operably (e.g., optically) connected to the optical assembly 420, and the second end of the optical fiber bundle 430 may be operably (e.g., optically) connected to the optical sensing device 440. In some embodiments, the first end of the optical fiber bundle 430 may be disposed in the scanning device 410, and the second end of the optical fiber bundle 430 may be disposed outside the scanning device 410. In some embodiments, if the optical assembly 420 is embedded into the wall of the scanning bore 411, the first end of the optical fiber bundle 430 and a segment of the optical fiber bundle 430 may be disposed inside a gantry of the scanning device 410. In some embodiments, if the optical assembly 420 hangs in the scanning bore 411, the first end of the optical fiber bundle 430 may be disposed in the scanning bore 411, while a segment of the optical fiber bundle 430 may be disposed inside the gantry of the scanning device 410. In some embodiments, if the optical assembly 420 is disposed on the scanning table 412, the first end of the optical fiber bundle 430 and/or a segment of the optical fiber bundle 430 may be embedded into the scanning table 412. The optical fiber bundle 430 may transmit the acquired optical signal from the optical assembly 420 to the optical sensing device 440. In some embodiments, the optical fiber bundle 430 may include a plurality of optical fibers. In some embodiments, an optical fiber may be of non-metallic materials including for example, glass, or plastic. In some embodiments, a number (or count) of the plurality of the optical fibers may be designed in order to satisfy an image resolution of a monitoring image. The number (or count) of the optical fibers may relate to the number (or count) of pixels of the monitoring image. For example, the number (or count) of the optical fibers may be equal to the number (or count) of pixels of the monitoring image. If a monitoring image of 256×256 pixels is desirable, then 65536 optical fibers may be needed in the optical fiber bundle 430. Each optical fiber may transmit an optical signal corresponding to a pixel of the monitoring image. If the number (or count) of the optical fibers is relatively large, the image resolution of the monitoring image may be relatively high.

The optical sensing device 440 may be configured to receive the optical signal and/or convert the optical signal to an electrical signal. The electrical signal may be used to generate a monitoring image. In some embodiments, the optical sensing device 440 may be a device with a function of photoelectric conversion. For example, the optical sensing device 440 may include but limited to a charge-coupled device (CCD), a complementary metal-oxide semiconductor (CMOS), or the like. In some embodiments, the optical sensing device 440 may be positioned outside the scanning device 410.

As shown in FIG. 4A, the image generator 450 may be operably connected to the optical sensing device 440. In some embodiments, the image generator 450 may be positioned outside the scanning device 410. The image generator 450 may be configured to generate a monitoring image (e.g., an image of the ROI) based on the received electrical signal. The generated monitoring image may be stored in a storage device (e.g., the storage device 150). In some embodiments, the image generator 450 may include a processing device (e.g., the computing device 200, or part of the processing device 140). For example, the processing device may include an analog-to-digital (AD) converter. The AD converter may convert the electrical signal to digital image data. In some embodiments, the image generator 450 may include a display device (e.g., a LED-based display). The generated monitoring image may be displayed on the display device. The status of the subject (e.g., whether the subject feels uncomfortable, or whether the subject moves or not) may be determined based on the monitoring image. Merely for illustration, if the monitoring image is an image of the head of the subject, the monitoring image may include facial information of the subject. Whether the subject feels uncomfortable may be determined according to the facial information. If it is found that the subject feels uncomfortable, a current scan may be suspended or adjusted to relieve the subject's uncomfortable feeling. For example, if the subject is found nervous by analyzing or observing a monitoring image generated at a moment, the processing device 140 or an operator of the scanning device 410 may take operations (e.g., playing a video on a display region within the subject' view) to relieve the subject's nervous feeling.

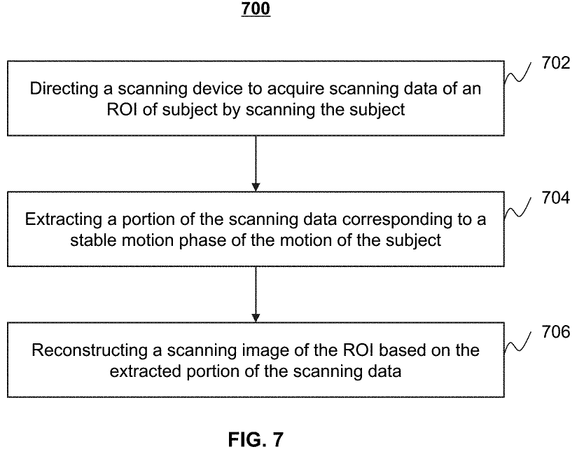

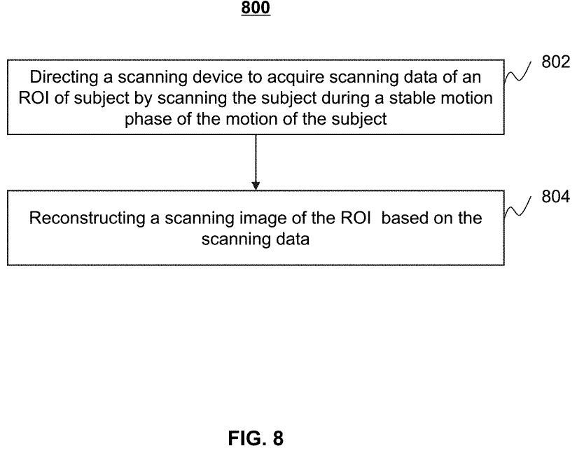

In some embodiments, a monitoring image may correspond to a moment (or a time point). Through the monitoring system 400a, a plurality of monitoring images may be generated. The plurality of monitoring images may correspond to different moments. In some embodiments, the plurality of monitoring images may be used to determine the motion of the subject (e.g., an organ motion or a posture change of the subject). The motion of the subject may bring image artifacts in a scanning image generated by the scanning device 410. In some embodiments, the image artifacts may be reduced or corrected based on the motion of the subject. For example, if it is found that the subject moves during the scan, the processing device 140 may perform one or more operations to avoid or reduce motion artifacts, and reconstruct a corrected scanning image. More descriptions about the reconstruction of the scanning image based on the motion of the subject may be found elsewhere in the present disclosure (e.g., FIGS. 7-8, and the descriptions thereof).

In some embodiments, the determination of the status of the subject may be performed automatically. For example, the processing device 140 may obtain one or more monitoring images from the image generator 450 or the storage device 150 (storing the one or more monitoring images). The processing device 140 may automatically identify one or more ROIs from the one or more monitoring images. The processing device 140 may analyze the status of the subject based on the ROIs. Merely by way of example, the processing device 140 may analyze one or more face expressions of the subject using one or more facial recognition techniques. Exemplary facial recognition techniques may include a local face analysis (LCA), a principal component analysis (PCA), neural networks (NN), a hidden Markov model, a linear discriminant analysis (LDA). The processing device 140 may determine whether the subject feels uncomfortable based on the face expression(s). In some embodiments, the processing device 140 may perform an automatic analysis of the status of the subject using one or more machine learning models (e.g., a convolutional neural network, a decision tree model, etc.).

Merely for illustration purposes, only one optical assembly 420 is illustrated in the monitoring system 400a. However, it should be noted that a plurality of optical assemblies 420 may be operably coupled to the scanning device 410. In some embodiments, two of the plurality of optical assemblies 420 may be designed to form a light reception device having a function of a binocular camera. Such light reception device may be used to capture 3D motion information of the subject and generate corresponding depth image. The generated depth image may be used to identify the motion of the subject. In some embodiments, the positions (e.g., heights, angles) of the plurality of optical assemblies 420 may be set according to the ROIs. For example, a first optical assembly may be mounted on a first position in the scanning device 410, and configured to acquire optical signal from the head. A second optical assembly may be mounted on a second position in the scanning device 410, and configured to acquire optical signal from the chest. The first position may be different from the second position. The plurality of optical assemblies 420 may be configured to acquire optical signals reflected or scattered by multiple ROIs of the subject. The acquired optical signals may be transmitted to the optical sensing device 440 through the optical fiber bundle 430 connected to thereof. The image generator 450 may generate multiple monitoring images corresponding to the multiple ROIs. According to the multiple monitoring images, the multiple ROIs of the subject may be monitored simultaneously. In some embodiments, the multiple monitoring images relating to the multiple ROIs may be registered and/or stitched to generate a comprehensive image of the subject. For example, the comprehensive image may illustrate a whole body of the subject. In some embodiments, the status of the subject may be monitored based on the multiple ROIs. For example, a user (e.g., an operator) may determine whether the subject feels uncomfortable by comprehensively analyzing the monitoring images of the head and the hands (the comprehensive image of the subject). In some embodiments, if there are a plurality of optical assemblies 420 in the monitoring system 400a, each optical assembly 420 may be operably connected to one optical fiber bundle 430, or at least part of the plurality of optical assemblies 420 may be operably connected to one optical fiber bundle 430. One or more optical fiber bundles 430 may be operably connected to the optical sensing device 440.

As described above, the subject may be placed in the examination space of the scanning bore 411 during the scan, the monitoring system 400a may monitor the status of the subject in real time or near real time. Specifically, the optical assembly 420 may acquire optical signal(s) reflected or scattered by the ROI of the subject. The optical signal(s) may be transmitted to the optical sensing device 440 through the optical fiber bundle 430. The optical sensing device 440 may transform the optical signal(s) to corresponding electrical signal(s). The image generator 450 may generate monitoring image(s) of the ROI(s) based on the electrical signal(s). A user (e.g., an operator) may observe, based on the monitoring image(s), the status of the subject in real time or near real time. Alternatively, the processing device 140 may automatically analyze the status of the subject based on the monitoring image(s). On the one hand, a relatively small number of components (e.g., the optical assembly 420, and/or a portion of the optical fiber bundle 430) may be disposed in the scanning device on the scanning table 412, and thus the examination space of the scanning bore 411 may not be greatly occupied. Therefore, the adjustment of position of the subject may not be affected by the components of the monitoring system. On the other hand, the optical assembly 420 and the optical fiber bundle 430 may be made of non-metallic materials, and no electricity may be needed or generated in the working process of the optical assembly 420 and the optical fiber bundle 430, thereby avoiding or reducing signal interferences with the scanning device 410 caused by metallic materials, and improving the quality of the scanning image(s) of the subject.

In some embodiments, the position of the optical sensing device 440 and the image generator 450 may be set according to a safety distance, respectively. For example, a distance (herein referred to as a first distance) between the optical sensing device 440 and the scanning device 410 may be greater than or equal to a first safety distance. A distance (herein referred to as a second distance) between the image generator 450 and the scanning device 410 may be greater than or equal to a second safety distance. The first safety distance and the second safety distance may refer to a critical distance at which the electricity in the optical sensing device 440 and the image generator 450 can't cause electromagnetic interference(s) to the scanning device 410, respectively. In some embodiments, the first and second safety distances may be determined based on an electromagnetic radiation power of the optical sensing device 440 and the image generator 450. For example, if the electromagnetic radiation power of the optical sensing device 440 is 40 W/m², the first safety distance may be set as 2 meters. If the electromagnetic radiation power of the image generator is 45 W/m², the first safety distance may be set as 2.5 meters. In some embodiments, the first safety distance and the second safety distance may be equal or different. For example, if the scanning device 410 is an MRI scanner, the first safety distance may be two meters and the second safety distance may be three meters. As another example, both the first safety distance and the second safety distance may be two meters.

In some embodiments, the optical fiber bundle 430 may be relatively long to make the optical sensing device 440 and the image generator 450 satisfy the first safety distance and the second safety distance, respectively. In some embodiments, a length of the optical fiber bundle 430 may be adjusted (or increased) such that the first distance or the second distance is greater than or equal to the first safety distance or the second safety distance. Therefore, electromagnetic interference(s) with a medical device that has a relatively high requirement for electromagnetic compatibility (e.g., an MRI scanner) may be reduced or avoided. In addition, the optical assembly 420 and the optical fiber bundle 430 may not need or generate electrical signal(s) and may not affect the operations of the MRI scanner. The optical assembly 420 and the optical fiber bundle 430 may be good compatible components for the MRI scanner. Besides, because the optical assembly 420 and the optical fiber bundle 430 are made of non-metallic materials, and the optical sensing device 440 and the image generator 450 satisfy the first safety distance and the second safety distance, the magnetic field generated by the MRI scanner may also not affect the normal operation of the optical assembly 420, the optical fiber bundle 430, the optical sensing device 440, and the image generator 450. Thereby the monitoring image generated by the image generator 450 may not be affected as well. A good image quality may be ensured, and the monitoring of the status of the subject may be facilitated.

According to the descriptions of FIG. 4A, for the monitoring system 400a, one optical fiber bundle 430 may be arranged in the scanning device 410, and may be optically coupled to the optical assembly 430 to transmit optical signal(s) from the optical assembly 430. It is unnecessary to arrange addition cables for the optical assembly 420, and the installation of the monitoring system can be facilitated. The optical fiber bundle 430 may not occupy much of the limited examination space of the scanning device 410, and the monitoring to the subject can be facilitated. Besides, the optical assembly 420 and the optical fiber bundle 430 are made of non-metallic materials, which may not bring image artifacts for the monitoring image. The image quality of the monitoring image may be improved.

FIG. 4B illustrates another exemplary monitoring system (a monitoring system 400b) for a medical device according to some embodiments of the present disclosure. Compared with the monitoring system 400a described in FIG. 4A, the monitoring system 400b may further include a first beam splitter 460 and a projector 470. The first beam splitter 460 may be disposed between the optical fiber bundle 430 and the optical sensing device 440. The projector 470 may be operably coupled to (e.g., optically connected to) the first beam splitter 460 (e.g., via optical connection means).

As illustrated in FIG. 4B, the optical assembly 420 may acquire an optical signal (herein referred to as a first optical signal) reflected or scattered by the ROI of the subject. The first optical signal may be transmitted from the first end of the optical fiber bundle 430 to the second end of the optical fiber bundle 430. The first beam splitter 460 may receive the first optical signal from the second end of the optical fiber bundle 430, and transmit the first optical signal to the optical sensing device 440. The optical sensing device 440 may convert the first optical signal to a corresponding electrical signal (herein referred to as a first electrical signal). The first electrical signal may be used to generate a monitoring image (herein referred to as a first image) by the image generator 450. In some embodiments, the projector 470 may acquire an electrical signal (herein referred to as a second electrical signal) associated with a second image. The second image may be obtained from a component of the imaging system 100 (e.g., the terminal 130, the processing device 140, the storage device 150, etc.). The second electrical signal may represent image data of the second image. For example, the second electrical signal may include an electrical signal corresponding to each pixel of the second image. In some embodiments, the second image may be used to relieve the subject's uncomfortable feelings (e.g., a funny image, or a video). In some embodiments, the second image may present an external environment of the scanning device 410, or a monitoring image (e.g., the first image). In some embodiments, the projector 470 may convert the second electrical signal to a second optical signal. In some embodiments, the projector 470 may transmit the second electrical signal to the first beam splitter 460. The second optical signal may be transmitted to the second end of the optical fiber bundle 430 through the first beam splitter 460. Then the second optical signal may be transmitted to the first end of the optical fiber bundle 430. In some embodiments, the projector 470 may include a device or circuits with a function of an electro-optic conversion, such as an electro-optic converter.

In some embodiments, the monitoring system 400b may also include a first polaroid (not shown in FIG. 4B). The first polaroid may be disposed between the first beam splitter 460 and the projector 470. The first polaroid may be configured to obtain or receive the second optical signal transmitted from a specified direction, and/or filter stray light(s). The first beam splitter 460 may perform a beam splitting for the first optical signal and/or the second optical signal. Merely by way of example, as shown in FIG. 4B, if there are two light beams desirable to pass through the first beam splitter 460, such as a light beam A and a light beam B, then the first beam splitter 460 may split the two light beams and transmit the two light beams to the optical sensing device 440 and the optical fiber bundle 430, respectively. As used herein, the light beam A represents the first optical signal acquired from the optical assembly 420, and the light beam B represents the second optical signal generated by the projector 470.

In some embodiments, a position of the first beam splitter 460 and/or a direction of beam splitting may be set according to positions of the optical sensing device 440 and/or the projector 470. For example, as shown in FIG. 4B, an incident direction of the light beam A may be perpendicular to the optical sensing device 440 through the first beam splitter 460. The light beam B may be reflected by the first beam splitter 460 at an angle (e.g., 45°), and be transmitted to the optical fiber bundle 430. Alternatively, the light beam A may be reflected by the first beam splitter 460 at an angle (e.g., 45°), and be transmitted to the optical sensing device 440. An incident direction of the light beam B may be perpendicular to the optical fiber bundle 430 through the first beam splitter 460.

In some embodiments, as shown in FIG. 4B, the monitoring system 400b may also include an optical transmission device 421 (e.g., including one or more optical lens). The optical transmission device 421 may be disposed between the optical fiber bundle 430 and the first beam splitter 460. The optical transmission device 421 may transmit the first optical signal from the second end of the optical fiber bundle 430 to the first beam splitter 460, and transmit the second optical signal from the first beam splitter 460 to the second end of the optical fiber bundle 430.

In some embodiments, as shown in FIG. 4B, the monitoring system 400b may further include a second beam splitter 480. The second beam splitter 480 may be configured to transmit the first optical signal from the optical assembly 420 to the first end of the optical fiber bundle 430, and transmit the second optical signal from the first end of the optical fiber bundle 430 to a display region.

In some embodiments, the second image relating to the second optical signal may be presented on the display region. In some embodiments, the display region may be set within a field of vison of the subject such that the subject can view the displayed second image. In some embodiments, the display region may be set inside the scanning bore 411. In some embodiments, the display region may be set in an external region of the scanning bore 411. In some embodiments, a display device may be disposed in the display region. As shown in FIG. 4B, a display device 490 may be disposed inside the scanning bore 411 and over the ROI of the subject. For example, if the ROI is the head of the subject, the display device 490 may be disposed over the head. The subject may view the second image displayed on the display device 490. The second image may be a static image or dynamic image. In some embodiments, the display device 490 may be omitted. The display region may include no additional display device (e.g., the display device 490), lest that the display device occupies the limited space of the scanning bore 411. The second optical signal may be directly projected on a surface (e.g., part of the inner wall of the scanning bore 411). That is, the display region may be directly set on the inner wall of the scanning bore 411. In some embodiments, the second optical signal transmitted by the second beam splitter 480 may be projected to the display region through one or more optical elements (not shown in FIG. 4B) between the second beam splitter 480 and the display region.

In some embodiments, the second beam splitter 480 may be configured to transmit an optical signal to a corresponding reception component. For example, the second beam splitter 480 may transmit the second optical signal (e.g., the light beam B) to the display region (e.g., the display device 490). As another example, the second beam splitter 480 may transmit the first optical signal to the first end of the optical fiber bundle 430.

A position of the second beam splitter 480 and/or a direction of beam splitting may be set according to positions of the optical assembly 420 and the display region (e.g., the display device 490). For example, as shown in FIG. 4B, an incident direction of the light beam A acquired by the optical assembly 420 may be perpendicular to the optical fiber bundle 430 through the second beam splitter 480. The light beam B may be reflected by the second beam splitter 480 at an angle (e.g., 45°), an be projected to the display region. Alternatively, the light beam A may be reflected by the second beam splitter 480 at an angle (e.g., 45°), and be transmitted to the optical fiber bundle 430. An incident direction of the light beam B may be perpendicular to the display region through the second beam splitter 480.

In some embodiments, as shown in FIG. 4B, the monitoring system 400b may further include a second optical transmission device 422 (e.g., including one or more optical lens). The second optical transmission device 422 may be disposed between the optical fiber bundle 430 and the second beam splitter 480. The second optical transmission device 422 may be configured to transmit the first optical signal from the second beam splitter 480 to the first end of the optical fiber bundle 430, and transmit the second optical signal from the first end of the optical fiber bundle 430 to the second beam splitter 480.

In some embodiments, the monitoring system 400b may further include a second polaroid (not shown in FIG. 4B). The second polaroid may be disposed between the second beam splitter 480 and the optical assembly 420. The second polaroid may be configured to obtain the first optical signal from a specified direction to facilitate the second beam splitter 460 to perform the beam splitting of the first optical signal.

As described above, the monitoring system 400b may perform bidirectional transmission of optical signals (e.g., the first optical signal and the second optical signal). On the one hand, the monitoring system 400b may transmit the first optical signal to an external device of the scanning bore 411, such as the optical sensing device 440, the image generator 450. The first optical signal may be used to generate the first image. The first image may be a monitoring image presenting the status of the subject in the examination space of the scanning bore 411. The subject may be monitored in real time or near real time according to the first image. On the other hand, the monitoring system 400b may transmit the second optical signal relating to the second image to the display region in the examination space of the scanning bore 411. The second image may be used to relieve uncomfortable feelings of the subject when the subject is viewing the second image. The second image may be a static image or a dynamic image (e.g., a video) for relieving the subject's feelings (e.g., nervous, painful, scared, etc.).

FIG. 4C illustrates another exemplary monitoring system (e.g., a monitoring system 400c) for a medical device according to some embodiments of the present disclosure. Compared with the monitoring system 400a described in FIG. 4A, the monitoring system 400c may further include a light source 491 and a fiber optic light guide 492. The light source 491 may be configured to provide a light. The fiber optic light guide 492 may be operably coupled to the light source 491. The fiber optic light guide 492 may transmit the light to the examination space of the scanning device 410 (e.g., inside the scanning bore 411). As shown in FIG. 4C, a first end of the fiber optic light guide 492 may be connected to the light source 491, and a second end of the fiber optic light guide 492 may be disposed inside the scanning bore 411. The fiber optic light guide 492 may be configured to transmit the light from the first end to the second end of the fiber optic light guide 492. The light may illuminate the ROI of the subject placed in the examination space in the scanning bore 411. When the light encounters the ROI of the subject, the light may be reflected or scattered. Then the optical assembly 420 may collect or acquire the optical signal corresponding to the reflected light or scattered light.

In some embodiments, the light source 491 may include but not limited to a white light source (e.g., an incandescent light bulb), a laser, a light emitting diode (LED), and so on. In some embodiments, the light source 491 may provide a narrowband light (e.g., a structured light) or a broadband light (e.g., a white light). For example, the structured light (e.g., an infrared light or an ultraviolet light) may be a three-dimensional (3D) structured light within a specified wavelength range. The structured light may be used to generate a depth image. In some embodiments, the structured light may have a predefined pattern. The predefined pattern may include a dot pattern, a stripe pattern, a checker board pattern, or the like, or any combination thereof. As shown in FIG. 4D, the predefined pattern may be a checker board pattern 495. If the structured light has the checker board pattern 495 and is projected on the ROI of the subject, the optical assembly 420 may acquire corresponding optical signal caused by the structured light. In some embodiments, due to the motion of the subject, the distortion of the pattern of the light acquired at different moments may be different. As shown in FIG. 4D, reference numeral 496 and reference numeral 497 may illustrate two exemplary patterns of the light acquired at two moments by the optical assembly 420. Compared with the predefined pattern (e.g., the pattern 495), the distortions of the pattern 496 and the pattern 497 may be different at the two moments. In some embodiments, the motion of the subject may be determined based on the distortions of patterns of light acquired at a plurality of moments.

In some embodiments, the light source 491 may be arranged such that a distance between the light source 491 and the scanning device 410 may be greater than or equal to a third safety distance (e.g., two meters). Accordingly, the light source 491 may not cause an electromagnetic interference with the scanning device 410, and the scanning device 410 may not cause an electromagnetic interference with the light source 491.

In some embodiments, the fiber optic light guide 492 may include a plurality of optical fibers. Each of the plurality of the optical fibers may transmit a beam let of the light to the scanning bore 411. If the brightness of the examination space in the scanning bore 411 is relatively low, the image quality of the generated monitoring image may be relatively poor. Therefore, it may be necessary to guarantee a satisfied brightness of the examination space in order to acquire the monitoring image with a relatively high quality. For example, if the light source 491 is working, the light source 491 may provide the light (e.g., a structured light) to illuminate the scanning bore 411 for improving the brightness of the examination space. In some embodiments, the light source 491 may adjust the intensity of the light according to the brightness of the examination space. In addition, in a functional magnetic resonance imaging (fMRI), the light may stimulate visuals of the subject so as to activate corresponding parts of the cerebral cortex of the subject, which can facilitate to locate a cortical central functional area and/or study brain function.

In some embodiments, as shown in FIG. 4C, the fiber optic light guide 492 and the optical fiber bundle 430 may be independent from each other. In some embodiments, the fiber optic light guide 492 may be integrated to the optical fiber bundle 430 in order to minimize their occupied examination space. For example, the fiber optic light guide 492 and the optical fiber bundle 430 may be disposed coaxially. In some embodiments, the fiber optic light guide 492 may be disposed around the optical fiber bundle 430. If the fiber optic light guide 492 is disposed around the optical fiber bundle 430, a relatively high light efficiency and/or a relatively high visual stimulation efficiency may be achieved.

In some embodiments, the monitoring system 400c may include a guide member 493 configured to facilitate a position adjustment of the optical assembly 420. As shown in FIG. 4C, the guide member 493 may be mounted on the inner wall of the scanning bore 411. In some embodiments, the guide member 493 may be mounted on the scanning table 412. The guide member 493 may guide the optical assembly 420 to move. With the movement of the optical assembly 420, the optical signal acquisition range of the optical assembly 420 may change accordingly, and the optical assembly 420 may acquire optical signals corresponding to different parts (or ROIs) of the subject.

In some embodiments, the guide member 493 may include a guide rail, and the optical assembly 420 may move along the guide rail. In some embodiments, the guide member 493 may have a curved guide rail or a straight guide rail, or a combination thereof. The guide member 493 may be made of a non-metallic material, such as glass, rubber or plastic. In some embodiments, the guide member 493 may be detachable. If there is no need to move the position of the optical assembly 420, the guide member 493 may be removed, thereby avoiding occupation of the examination space. It should be note that a length and/or a size of the guide member 493 may not be intended to be limiting in the present disclosure. The guide member 493 may be set on any suitable position of the inner wall of the scanning bore 411 or the scanning table 412 so that the optical assembly 420 may move along the guide member 493 to acquire the optical signal corresponding to the ROI of the subject. For example, the position of the guide member 493 may be set according to a desired observation region (e.g., a whole examination space of the scanning bore 113), so that the optical assembly 420 can move to any position of the desired observation region along the guide member 493, and acquire corresponding optical signal(s). Therefore, the status of the subject can be monitored from various viewing angles, and the monitoring may be facilitated.

In some embodiments, the monitoring system 400c may further include a lifting device (not shown in FIG. 4C) mounted on the inner wall of the scanning bore 411 or the scanning table 412. The lifting device may be configured to change the height of the optical assembly 420. It should be noted that the position of the lifting device may not be intended to be limiting. The optical assembly 420 may be operably connected to the lifting device. The lifting device may adjust the height of the optical assembly 420 in order to adjust the optical signal acquisition range of the optical assembly 420 and/or a viewing angle of the optical assembly 420. The optical assembly 420 may acquire the optical signal from the viewing angle. In some embodiments, the lifting device may be detachable. If there is no need to adjust the height of the optical assembly 420, the lifting device can be removed, thereby avoiding occupation of the examination space. The lifting device may be set on any suitable position of the inner wall of the scanning bore 411 or the scanning table 412, so that the height of the optical assembly 430 may be adjusted to acquire the optical signal corresponding to the ROI of the subject.

It should be noted that the above descriptions of the monitoring systems 400a, 400b and 400c are merely provided for the purposes of illustration, and not intended to limit the scope of the present disclosure. For persons having ordinary skills in the art, multiple variations and modifications may be made under the teachings of the present disclosure. For example, the monitoring system may include a portion of components of one of the monitoring system 400a, 400b or 400c. As another example, the monitoring system may include all components of one or more of the monitoring systems 400a, 400b and 400c. As a further example, one or more of the monitoring systems 400a, 400b and 400c may be combined with the components of another monitoring system.

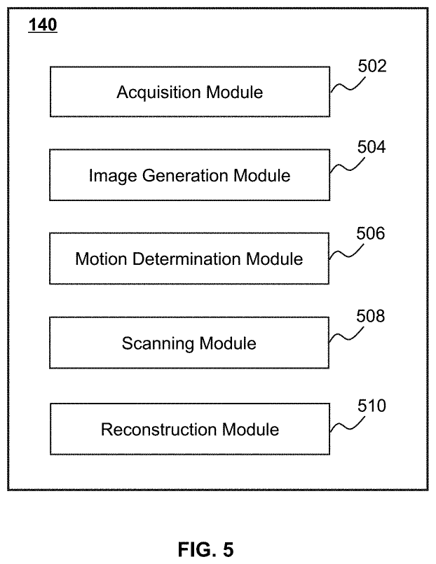

FIG. 5 is a block diagram illustrating an exemplary processing device according to some embodiments of the present disclosure. The processing device 140 may include an acquisition module 502, an image generation module 504, a motion determination module 506, a scanning module 508, and a reconstruction module 510. At least a portion of the processing device 140 may be implemented on computing device 200 as illustrated in FIG. 2 or mobile device 300 as illustrated in FIG. 3.

The acquisition module 502 may be configured to acquire optical signal(s), electrical signal(s), and/or image(s) of the ROI of the subject. In some embodiments, the acquisition module 502 may acquire optical signals of the ROI from the optical fiber bundle 430. In some embodiments, the acquisition module 502 may acquire electrical signals of the ROI from the optical sensing device 440. In some embodiments, the acquisition module 502 may acquire images of the ROI from the image generator 450. In some embodiments, the ROI of the subject may include an organ (e.g., a lung, the liver, the stomach), a body part (e.g., the chest, the abdomen, the head), an injured part, a tumor, or the like, or any combination thereof. In some embodiments, the acquisition module 502 may cause the light source 491 to provide a light to illuminate the examination space in the scanning bore 411. When the light encounters the ROI of the subject, the optical assembly 420 may be specified to acquire the optical signal reflected or scattered by the ROI of the subject. In some embodiments, the light may be a structured light having a predefined pattern (e.g., the checker board pattern 495 shown in FIG. 4D). The acquired optical signal may be the structured light reflected or scattered by the ROI of the subject.

In some embodiments, the optical signal may be transmitted to the optical sensing device 440 through an optical fiber bundle 430. The image generation module 504 may be configured to cause the optical sensing device 440 to perform the photoelectric conversion operation. Then the optical signal may be converted to corresponding electrical signal. The image generation module 504 may be configured to cause the image generator 450 to generate the monitoring image based on the electrical signal. In some embodiments, the image generation module 504 may generate the monitoring image based on the electrical signal(s) acquired by the acquisition module 502. In some embodiments, the generated monitoring image may be stored in a storage device (e.g., the storage device 150). In some embodiments, the monitoring image may be presented on a display device. In some embodiments, the display device may be part of the image generator 450. In some embodiments, the display device may be an external device connected to the image generator 450. The status of the subject (e.g., whether the subject feels uncomfortable, or whether the subject moves or not) may be monitored based on the monitoring image.

The motion determination module 506 may be configured to determine the motion of the subject based on the monitoring images at different moments. In some embodiments, the motion of the subject may include a physiological motion (e.g., a cardiac motion or a respiratory motion) of the subject and/or a posture change of the subject. In some embodiments, the acquisition module 502 may obtain a plurality of monitoring images at different moments. The motion determination module 506 may obtain the plurality of monitoring images from the acquisition module 502.

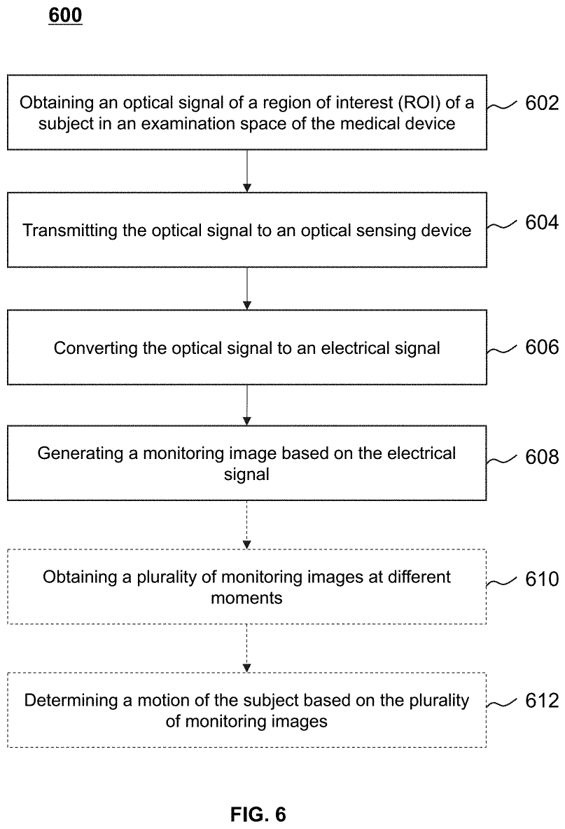

As described in connection with FIG. 4C, in some embodiments, the light source 491 may provide the structured light, and transmit, via the fiber optic light guide 492, the structured light. The fiber optic light guide 492 may project the structured light onto the ROI of the subject. The structured light may be a light encoded with the predefined pattern (e.g., the checker board pattern 495 shown in FIG. 4D). Due to the motion of the subject, different positions on the subject may have different heights, the pattern of the light corresponding to the optical signal acquired by the optical assembly 420 may be distorted compared to the structured light from the light source 491, for example, the pattern of the light 496 and the pattern of the light 497 shown in FIG. 4D, they are the distorted patterns of light. Undergoing the motion of the subject (e.g., the cardiac motion, the respiratory motion, or the posture change), a distortion of the pattern of the light acquired at different time points may be different. One monitoring image may correspond to a moment of the motion of the subject. In some embodiments, the motion determination module 506 may designate a monitoring image with the smallest distortion among the plurality of monitoring images as a reference image. The reference image and the plurality of monitoring images may be used to determine the motion of the subject. More descriptions regarding the determination of the motion of the subject may be found elsewhere in the present disclosure (e.g., FIG. 6, and the descriptions thereof).

The scanning module 508 may be configured to acquire scanning data of the ROI of the subject by scanning the subject. In some embodiments, the scanning module 508 may cause the scanning device to scan the subject based on a stable motion phase of the motion of the object. The scanning data may be generated. In some embodiments, the scanning module 508 may extract a portion of the scanning data corresponding to the stable motion phase of the motion of the subject.

The reconstruction module 510 may be configured to the reconstruct the scanning image based on at least a portion of the scanning data. In some embodiments, the reconstruction module 510 may reconstruct the image according to a reconstruction technique. Exemplary reconstruction techniques may include but are not limited to an algebraic reconstruction technique (ART), a simultaneous algebra reconstruction technique (SART), a filtered back projection (FBP) technique, a Feldkamp-Davis-Kress (FDK) reconstruction technique, an iterative reconstruction technique, a convolution back projection (CBP) technique, a Fourier back projection technique, or the like, or any combination thereof.

The modules in the processing device 140 may be connected to or communicate with each other via a wired connection or a wireless connection. The wired connection may include a metal cable, an optical cable, a hybrid cable, or the like, or any combination thereof. The wireless connection may include a Local Area Network (LAN), a Wide Area Network (WAN), a Bluetooth, a ZigBee, a Near Field Communication (NFC), or the like, or any combination thereof.

It should be noted that the above description is merely provided for the purposes of illustration, and not intended to limit the scope of the present disclosure. For persons having ordinary skills in the art, multiple variations and modifications may be made under the teachings of the present disclosure. However, those variations and modifications do not depart from the scope of the present disclosure. For example, the processing device 140 may further include a storage module (not shown in FIG. 5). The storage module may be configured to store data generated by the processing device 140.

FIG. 6 is a flowchart illustrating an exemplary monitoring process according to some embodiments of the present disclosure. In some embodiments, process 600 may be implemented in the imaging system 100 illustrated in FIG. 1 through the monitoring system 400a, 400b, or 400c illustrated in FIGS. 4A-4C. The monitoring system 400a, 400b, or 400c may be part of the imaging system 100. The process 600 may be stored in a storage device (e.g., the storage device 150, the storage 220, or the storage 390) as a form of instructions, and can be invoked and/or executed by the processing device 140 (e.g., the processor 210 of the computing device 200, or one or more modules in the processing device 140 illustrated in FIG. 5). The operations of the illustrated process 600 presented below are intended to be illustrative. In some embodiments, the process 600 may be accomplished with one or more additional operations not described, and/or without one or more of the operations discussed. Additionally, the order in which the operations of the process 600 as illustrated in FIG. 6 and described below is not intended to be limiting.

In 602, an optical signal of a region of interest (ROI) of a subject positioned in an examination space of the medical device may be obtained. For example, the optical assembly 420 may be configured to acquire the optical signal of the ROI of the subject. In some embodiments, the acquisition module 502 may obtain the optical signal acquired by the optical assembly 420. In some embodiments, the medical device may include a scanning device (e.g., the scanning device 110 or the scanning device 410) for scanning the ROI of the subject. The scanning device may include an MRI scanner, a PET scanner, a CT scanner, or the like, or any combination thereof. The examination space of the scanning device may accommodate the subject to be scanned. The examination space may refer to the space surrounded by a scanning bore (e.g., the scanning bore 411) of the scanning device (e.g., the scanning device 410). In some embodiments, the ROI of the subject may include an organ (e.g., a lung, the liver, the stomach), a body part (e.g., the chest, the abdomen, the head), an injured part, a tumor, or the like, or any combination thereof.

In some cases, if the brightness of the examination space is relatively low, the acquired optical signal may be weak resulting in a poor image quality. In some embodiments, the processing device 140 may direct an external light source (e.g., the light source 491) to provide a light to illuminate the examination space. The acquired optical signal may be improved accordingly. In some embodiments, the light may be transmitted into the examination space through a fiber optic light guide (e.g., the fiber optic light guide 492). When the light encounters the ROI of the subject, the optical assembly 420 may acquire the optical signal reflected or scattered by the ROI of the subject.

Merely by way of example, the light source 491 may provide a narrowband light (e.g., a structured light) or a broadband light (e.g., a white light). For example, the light source 491 may provide a structured light having a predefined pattern (e.g., the checker board pattern 495). In some embodiments, the structured light may be used to generate a depth image. The depth image may be further used to identify the motion of the subject. Upon receipt of the structured light from the light source 491, the fiber optic light guide 492 may transmit and project the structured light onto the ROI (e.g., the chest and abdomen of the subject) of the subject. The optical assembly 420 may acquire or collect the optical signal resulting from the structured light projected onto the ROI. In some embodiments, optical signals acquired at different moments may correspond to different patterns of light (e.g., pattern 496 at a first moment and pattern 497 at a second moment).

In 604, the acquired optical signal may be transmitted to an optical sensing device (e.g., the optical sensing device 440) through an optical fiber bundle (e.g., the optical fiber bundle 430). Merely by way of example, as described in connection with FIGS. 4A-4C, a first end of the optical fiber bundle 430 may be operably connected to the optical assembly 420, and a second end of the optical fiber bundle 430 may be operably connected to the optical sensing device 440. The optical fiber bundle 430 may transmit the optical signal to the optical sensing device 440.

In some embodiments, the optical fiber bundle may include a plurality of optical fibers. In some embodiments, the optical fibers may be made of non-metallic materials including for example, plastic, or glass. In some embodiments, a number (or count) of the plurality of the optical fibers may be designed in order to satisfy an image resolution of a monitoring image. The number (or count) of the optical fibers may relate to the number (or count) of pixels of the monitoring image. For example, the number (or counts) of the optical fibers may be equal to the number (or count) of pixels of the monitoring image. If a monitoring image of 256×256 pixels is desirable, then 65536 optical fibers may be needed in the optical fiber bundle 430. Each optical fiber may transmit an optical signal corresponding to a pixel of the monitoring image. If the number (or count) of the optical fibers is relatively large, the image resolution of the monitoring image may be relatively high.

In 606, the optical signal may be converted to an electrical signal. For example, the processing device 140 may cause the optical sensing device 440 to perform the photoelectric conversion operation. Upon receipt of the optical signal, the optical sensing device 440 may convert the optical signal to a corresponding electrical signal. The electrical signal may be used to generate a monitoring image. In some embodiments, the optical sensing device 440 may be a device with a function of photoelectric conversion. For example, the optical sensing device 440 may include but limited to a charge-coupled device (CCD), a complementary metal-oxide semiconductor (CMOS), or the like.

In 608, a monitoring image may be generated based on the electrical signal. For example, the image generation module 504 of the processing device 140 may cause the image generator 450 to generate the monitoring image based on the electrical signal. In some embodiments, the generated monitoring image may be stored in a storage device (e.g., the storage device 150). In some embodiments, the monitoring image may be presented on a display device. In some embodiments, the display device may be part of the image generator 450. In some embodiments, the display device may be an external device coupled to the image generator 450. The status of the subject (e.g., whether the subject feels uncomfortable, or whether the subject moves or not) may be determined based on the monitoring image.

Merely for illustration, if the monitoring image is an image of the head of the subject, the monitoring image may include facial information of the subject. Whether the subject feels uncomfortable may be determined according to the facial information. If it is found that the subject feels uncomfortable, a current scan may be suspended or adjusted to relieve the subject's uncomfortable feeling(s). For example, if the subject is found nervous by analyzing or observing a monitoring image generated at a moment, the processing device 140 or an operator of the scanning device 140 may take operations (e.g., playing a video on a display region within the subject' view) to relieve the subject's nervous feeling. In some embodiments, the display region may include a specified display device (e.g., the display device 490) disposed in the examination space. In some embodiments, the display region may be part of the inner wall of the scanning bore 411 and the display device may be omitted.

In some embodiments, the occurrence of the motion of the subject may bring image artifacts in a scanning image. The scanning image may be reconstructed based on scanning data acquired by the scanning device (e.g., the scanning device 410). In some embodiments, the generated monitoring image(s) may be further used to determine the motion of the subject occurred during the scan. In some embodiments, the motion of the subject may be determined using one or more image recognition techniques described in the present disclosure. In some embodiments, the image artifacts of the scanning image(s) may be reduced or removed based on the motion related data. In some embodiments, the motion of the subject may be continuously motioned in a time period (e.g., during the scanning of the scanning device 410). Operations 610 and 612 may be performed to determine the motion(s) of the subject based on the generated monitoring image(s).

In 610, a plurality of monitoring images at different moments may be obtained. In some embodiments, operation 610 may be performed by the acquisition module 502 of the processing device 140. In some embodiments, a monitoring image may correspond to a moment (or a time point). A plurality of monitoring images at different moments may be generated by the monitoring system (e.g., the monitoring system 400a, 400b, or 400c) described in the present disclosure. The plurality of monitoring images may be stored in a storage device (e.g., the storage device 150). The acquisition module 502 may acquire the plurality of monitoring image from the monitoring system (e.g., the image generator 450) or the storage device 150. In some embodiments, the image generation module 504 may obtain electrical signals of the ROI of the subject (e.g., from the optical sensing device 440) and generate the monitoring image(s).

In 612, the motion of the subject may be determined based on the plurality of monitoring images. In some embodiments, the operation 612 may be performed by the motion determination module 506 of the processing device 140. In some embodiments, the motion of the subject may include a physiological motion (e.g., a cardiac motion or a respiratory motion) of the subject and/or a posture change of the subject.

In some embodiments, the processing device 140 may determine, based on the plurality of monitoring images at different moments, the motion of the subject using a motion detection algorithm. Exemplary motion detection algorithms may include a background subtraction algorithm, an optical flow algorithm, an active contour model based tracking algorithm, a continuously adaptive Mean-SHIFT algorithm, a machine learning algorithm (e.g., neural networks), or the like, or any combination thereof. The motion related data may be used to remove or reduce a motion artifact of a scanning image.

As described in connection with FIG. 4C, in some embodiments, the light source 491 may provide the structured light, and transmit, via the fiber optic light guide 492, the structured light. The fiber optic light guide 492 may project the structured light onto the ROI of the subject. The structured light may be a light encoded with the predefined pattern (e.g., the checker board pattern 495 shown in FIG. 4D). Due to the motion of the subject, different portions on the subject may have different heights, the pattern of the light corresponding to the optical signal acquired by the optical assembly 420 may be distorted compared to the structured light from the light source 491. For example, the pattern of the light 496 and the pattern of the light 497 shown in FIG. 4D may be exemplary distorted patterns of light. If a motion of the subject (e.g., the cardiac motion, the respiratory motion, or the posture change) occurs, a distortion of the pattern of the light may be presented, and distortions acquired at different time points may be different. One monitoring image may correspond to a moment of the motion of the subject. In some embodiments, the processing device 140 may designate a monitoring image with a minimum distortion among the plurality of monitoring images as a reference image. In some embodiments, the processing device 140 may designate an initial image (e.g., a first image generated at a first moment when the monitoring system starts to monitor the subject) as the reference image. In some embodiments, the processing device 140 may designate a static image generated when the subject is static as the reference image. The reference image and the plurality of monitoring images may be used to determine the motion of the subject.

In some embodiments, the plurality of monitoring images and the reference image may correspond to the ROI of the subject. Specifically, each of the plurality of monitoring images may include a plurality of pixels corresponding to a plurality of portions of the ROI. The reference image may also include a plurality of pixels corresponding to the plurality of portions of the ROI. For each monitoring image, the processing device 140 may determine a plurality of measured distances based on the monitoring image and a geometrical relationship between the fiber optic light guide 492 and the optical assembly 420. In some embodiments, the geometrical relationship may be characterized by at least one of a distance between the second end of the fiber optic light guide 492 (that is, the end disposed in the examination space) and the optical assembly 420, an orientation of the second end of the fiber optic light guide 492, an orientation of the optical assembly 420, or the like, or any combination thereof. A measured distance may be a distance between one of the plurality of portions of the ROI as represented in the monitoring image and the second end of the fiber optic light guide 492. The processing device 140 may determine a plurality of reference distances based on the geometrical relationship and the reference image. A reference distance may be a distance between one of the plurality of portions of the ROI as represented in the reference image and the second end of the fiber optic light guide 492. The processing device 140 may determine a depth image corresponding to the monitoring image based on a plurality of differences. Each of the plurality of differences may be a difference between one of the plurality of measured distances of a portion of the ROI and a reference distance of the same portion of the ROI. Similarly, a plurality of depth images corresponding to the plurality of monitoring images may be determined. The processing device 140 may determine the motion of the subject based on the plurality of depth images corresponding to the plurality of monitoring images. For example, the processing device 140 may analyze the changes of the depth images at different moments (or time points) to determine the motion of the subject. More descriptions regarding the determination of the motion may be found in, e.g., U.S. patent application Ser. No. 16/510,254 entitled “SYSTEMS AND METHODS FOR DETERMINING MOTION OF AN OBJECT IN IMAGING,” filed on Jul. 12, 2019.

It should be noted that the above description is merely provided for the purposes of illustration, and not intended to limit the scope of the present disclosure. For persons having ordinary skills in the art, multiple variations and modifications may be made under the teachings of the present disclosure. However, those variations and modifications do not depart from the scope of the present disclosure. For example, operations 610 and 612 may be integrated into a single operation. As another example, operations 610 and 612 may be omitted.

FIG. 7 is a flowchart illustrating an exemplary process for reconstructing a scanning image of a subject according to some embodiments of the present disclosure. In some embodiments, process 700 may be implemented in the imaging system 100 illustrated in FIG. 1 including the monitoring system 400a, 400b, or 400c illustrated in FIGS. 4A-4C. The process 700 may be stored in a storage device (e.g., the storage device 150, the storage 220, or the storage 390) as a form of instructions, and can be invoked and/or executed by the processing device 140 (e.g., the processor 210 of the computing device 200, or one or more modules in the processing device 140 illustrated in FIG. 5). The operations of the illustrated process 700 presented below are intended to be illustrative. In some embodiments, the process 700 may be accomplished with one or more additional operations not described, and/or without one or more of the operations discussed. Additionally, the order in which the operations of the process 700 as illustrated in FIG. 7 and described below is not intended to be limiting.

In 702, the processing device 140 (e.g., the scanning module 508 of the processing device 140) may direct a scanning device (e.g., the scanning device 110 or the scanning device 410) to acquire scanning data of an ROI of the subject by scanning the subject. The ROI of the subject may include an organ (e.g., a lung, the liver, the stomach), a body part (e.g., the chest, the abdomen, the head), an injured part, a tumor, or the like, or any combination thereof.

Specifically, when the ROI of the subject is moved into the examination space of the scanning device, the processing device 140 may direct the scanning device to scan the ROI according to a scanning protocol. The scanning protocol may include a plurality of scanning parameter(s) and/or reconstruction parameter(s). For example, for MR scanning, the scanning protocol may include but not limited to a repetition time (TR), an echo time (TE), an inversion time (TI), the number of excitation (NEX), an acquisition time (TA), a slice thickness, a slice gap, a matrix, the field of view (FOV), a flip angle, or the like, or any combination thereof. For CT scanning, the scanning parameters may include a scanning type, a tube voltage, a tube current, a scanning time, a field of view, a matrix, a collimation, an acquisition channel, a slice thickness, a slice gap, a pitch, a rotation speed, a cardiac gating, a reconstruction algorithm, or the like, or any combination thereof.

In some embodiments, the processing device 140 may simultaneously or synchronously perform the monitoring procedure using one or more components (e.g., the optical assembly 420, the optical sensing device 440, the image generator 450, etc.) of the monitoring system and direct the scanning device (e.g., the scanning device 110 or 410) to acquire scanning data of the subject. In some embodiments, the scanning data may correspond to one or more phases of the motion of the subject. The determination of the motion of the subject may refer to operation 610 described in FIG. 6. In some embodiments, the motion may include a relatively stable motion phase (e.g., the mid and late diastole, the expiratory phase, the eye opening phase) and a relatively unstable motion phase (e.g., the systole, the inspiratory phase, the eye blinking phase). A portion of the scanning data may correspond to the relatively stable motion phase of the motion, while another portion of the scanning data may correspond to the relatively unstable motion phase of the motion.

In 704, the processing device 140 (e.g., the scanning module 508 of the processing device 140) may extract the portion of the scanning data corresponding to the stable motion phase of the motion. In some embodiments, the processing device 140 may determine the stable motion phase of the motion and determine a portion of the scanning data corresponding to the stable motion phase. The processing device 140 may then extract the portion of the scanning data. The extracted portion of the scanning data may be barely affected by the motion of the object.

In 706, the processing device 140 (e.g., the reconstruction module 510 of the processing device 140) may reconstruct a scanning image of the ROI based on the extracted portion of the scanning data. In some embodiments, the reconstructed image may be a two-dimensional (2D) image or a three-dimensional (3D) image. In some embodiments, the processing device 140 may reconstruct the image according to a reconstruction technique. Exemplary reconstruction techniques may include but are not limited to an algebraic reconstruction technique (ART), a simultaneous algebra reconstruction technique (SART), a filtered back projection (FBP) technique, a Feldkamp-Davis-Kress (FDK) reconstruction technique, an iterative reconstruction technique, a convolution back projection (CBP) technique, a Fourier back projection technique, or the like, or any combination thereof.

It should be understood that according to the reconstruction of the scanning image of the ROI using the scanning data acquired or obtained when the motion of the object is relatively stable, motion artifacts in the reconstructed image may be effectively reduced or removed.

In some embodiments, the processing device 140 may adjust or update scanning parameters based on the determined motion related data. For example, during the MRI scan, if the head of the subject is moved, the monitoring system may monitor the motion of the head in real time or near real time. The motion related data may be determined based on the monitoring images generated by the monitoring system. The motion related data may include motion parameters related to the motion of the head. The motion parameters may include a translation matrix and/or rotation matrix. In some embodiments, the processing device 140 may adjust the scanning parameters based on the motion parameters, such as gradient field parameters, Gx, Gy or Gz. The MR scanner may scan the subject based on the adjusted scanning parameters in order to reduce motion artifacts caused by the head motion. The scanning data may be obtained accordingly. The processing device 140 may reconstruct an image based on the obtained scanning data.

FIG. 8 is a flowchart illustrating an exemplary process for reconstructing a scanning image of a subject according to some embodiments of the present disclosure. In some embodiments, process 800 may be implemented in the imaging system 100 illustrated in FIG. 1 including the monitoring system 400a, 400b, or 400c illustrated in FIGS. 4A-4C. The process 800 may be stored in a storage device (e.g., the storage device 150, the storage 220, or the storage 390) as a form of instructions, and can be invoked and/or executed by a processing device (e.g., the processor 210 of the computing device 200, or one or more modules in the processing device 140 illustrated in FIG. 5). The operations of the illustrated process 800 presented below are intended to be illustrative. In some embodiments, the process 800 may be accomplished with one or more additional operations not described, and/or without one or more of the operations discussed. Additionally, the order in which the operations of the process 800 as illustrated in FIG. 8 and described below is not intended to be limiting.

In 802, the processing device 140 (e.g., the scanning module 508 of the processing device 140) may direct a scanning device (e.g., the scanning device 110 or the scanning device 410) to acquire scanning data of an ROI of the subject by scanning the subject during the stable motion phase of the motion. The ROI of the subject may include an organ (e.g., a lung, the liver, the stomach), a body part (e.g., the chest, the abdomen, the head), an injured part, a tumor, or the like, or any combination thereof.

In some embodiments, the status of the subject may be monitored in real time or near real time according to the monitoring images generated at different moments. In some embodiments, the processing device 140 may determine the motion of the subject based on the monitoring images synchronously. The motion may include a relatively stable motion phase (e.g., the mid and late diastole, the expiratory phase, the eye opening phase) and a relatively unstable motion phase (e.g., the systole, the inspiratory phase, the eye blinking phase). In some embodiments, the processing device 140 may trigger the scanning device to acquire the scanning data of the ROI based on the motion of the subject. For example, when the motion enters into the relatively stable motion phase (e.g., after a falling edge of a period diagram of the motion), the processing device 140 may trigger the scanning device to acquire scanning data of the subject. As another example, when the motion enters into the relatively unstable motion phase (e.g., before a rising edge of the period diagram of a physiological motion), the processing device 140 may direct the scanning device 410 to stop acquiring scanning data of the subject. Thus, the scanning data acquired by the scanning device may be barely affected by the motion of the subject.

In 804, the processing device 140 (e.g., the reconstruction module 510 of the processing device 140) may reconstruct a scanning image of the ROI based on the scanning data. In some embodiments, the scanning image may be a two-dimensional (2D) image or a three-dimensional (3D) image. In some embodiments, the processing device 140 may reconstruct the scanning image according to a reconstruction technique. Exemplary reconstruction techniques may include but are not limited to an algebraic reconstruction technique (ART), a simultaneous algebra reconstruction technique (SART), a filtered back projection (FBP) technique, a Feldkamp-Davis-Kress (FDK) reconstruction technique, an iterative reconstruction technique, a convolution back projection (CBP) technique, a Fourier back projection technique, or the like, or any combination thereof.

Having thus described the basic concepts, it may be rather apparent to those skilled in the art after reading this detailed disclosure that the foregoing detailed disclosure is intended to be presented by way of example only and is not limiting. Various alterations, improvements, and modifications may occur and are intended to those skilled in the art, though not expressly stated herein. These alterations, improvements, and modifications are intended to be suggested by this disclosure, and are within the spirit and scope of the exemplary embodiments of this disclosure.

Moreover, certain terminology has been used to describe embodiments of the present disclosure. For example, the terms “one embodiment,” “an embodiment,” and/or “some embodiments” mean that a particular feature, structure or characteristic described in connection with the embodiment is included in at least one embodiment of the present disclosure. Therefore, it is emphasized and should be appreciated that two or more references to “an embodiment” or “one embodiment” or “an alternative embodiment” in various portions of this specification are not necessarily all referring to the same embodiment. Furthermore, the particular features, structures or characteristics may be combined as suitable in one or more embodiments of the present disclosure.

Further, it will be appreciated by one skilled in the art, aspects of the present disclosure may be illustrated and described herein in any of a number of patentable classes or context including any new and useful process, machine, manufacture, or composition of matter, or any new and useful improvement thereof. Accordingly, aspects of the present disclosure may be implemented entirely hardware, entirely software (including firmware, resident software, micro-code, etc.) or combining software and hardware implementation that may all generally be referred to herein as a “unit,” “module,” or “system.” Furthermore, aspects of the present disclosure may take the form of a computer program product embodied in one or more computer-readable media having computer readable program code embodied thereon.

A computer readable signal medium may include a propagated data signal with computer readable program code embodied therein, for example, in baseband or as part of a carrier wave. Such a propagated signal may take any of a variety of forms, including electromagnetic, optical, or the like, or any suitable combination thereof. A computer readable signal medium may be any computer readable medium that is not a computer readable storage medium and that may communicate, propagate, or transport a program for use by or in connection with an instruction execution system, apparatus, or device. Program code embodied on a computer readable signal medium may be transmitted using any appropriate medium, including wireless, wireline, optical fiber cable, RF, or the like, or any suitable combination of the foregoing.

Computer program code for carrying out operations for aspects of the present disclosure may be written in any combination of one or more programming languages, including an object-oriented programming language such as Java, Scala, Smalltalk, Eiffel, JADE, Emerald, C++, C#, VB. NET, Python or the like, conventional procedural programming languages, such as the “C” programming language, Visual Basic, Fortran 2103, Perl, COBOL 2102, PHP, ABAP, dynamic programming languages such as Python, Ruby, and Groovy, or other programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider) or in a cloud computing environment or offered as a service such as a Software as a Service (SaaS).

Furthermore, the recited order of processing elements or sequences, or the use of numbers, letters, or other designations, therefore, is not intended to limit the claimed processes and methods to any order except as may be specified in the claims. Although the above disclosure discusses through various examples what is currently considered to be a variety of useful embodiments of the disclosure, it is to be understood that such detail is solely for that purpose, and that the appended claims are not limited to the disclosed embodiments, but, on the contrary, are intended to cover modifications and equivalent arrangements that are within the spirit and scope of the disclosed embodiments. For example, although the implementation of various components described above may be embodied in a hardware device, it may also be implemented as a software-only solution, for example, an installation on an existing server or mobile device.

Similarly, it should be appreciated that in the foregoing description of embodiments of the present disclosure, various features are sometimes grouped together in a single embodiment, figure, or description thereof for the purpose of streamlining the disclosure aiding in the understanding of one or more of the various inventive embodiments. This method of disclosure, however, is not to be interpreted as reflecting an intention that the claimed subject matter requires more features than are expressly recited in each claim. Rather, inventive embodiments lie in less than all features of a single foregoing disclosed embodiment.

In some embodiments, the numbers expressing quantities or properties used to describe and claim certain embodiments of the application are to be understood as being modified in some instances by the term “about,” “approximate,” or “substantially.” For example, “about,” “approximate,” or “substantially” may indicate ±20% variation of the value it describes, unless otherwise stated. Accordingly, in some embodiments, the numerical parameters set forth in the written description and attached claims are approximations that may vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the application are approximations, the numerical values set forth in the specific examples are reported as precisely as practicable.

Each of the patents, patent applications, publications of patent applications, and other material, such as articles, books, specifications, publications, documents, things, and/or the like, referenced herein is hereby incorporated herein by this reference in its entirety for all purposes, excepting any prosecution file history associated with same, any of same that is inconsistent with or in conflict with the present document, or any of same that may have a limiting affect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

In closing, it is to be understood that the embodiments of the application disclosed herein are illustrative of the principles of the embodiments of the application. Other modifications that may be employed may be within the scope of the application. Thus, by way of example, but not of limitation, alternative configurations of the embodiments of the application may be utilized in accordance with the teachings herein. Accordingly, embodiments of the present application are not limited to that precisely as shown and described.

1. A system, comprising:
an optical assembly configured to acquire an optical signal associated with a region of interest (ROI) of a subject in an examination space of a medical device;
an optical fiber bundle operably connected to the optical assembly, and configured to transmit the optical signal;
an optical sensing device operably connected to the optical fiber bundle, and configured to receive the optical signal and convert the optical signal to an electrical signal; and
an image generator operably connected to the optical sensing device, and configured to generate, based on the electrical signal, a monitoring image of the ROI.

2. The system of claim 1, wherein the optical assembly is operably located in the examination space of the medical device, or is movable in or to the examination space of the medical device.

3. The system of claim 1, wherein the optical sensing device and the image generator are disposed outside the medical device, a first distance between the optical sensing device and the medical device being greater than a first safety distance and a second distance between the image generator and the medical device being greater than a second safety distance.

4. (canceled)

5. The system of claim 3, wherein the first safety distance is set such that a working current of the optical sensing device has no effect on a working condition of the medical device, and the second safety distance is set such that a working current of the image generator has no effect on a working condition of the medical device.

6. The system of claim 1, further comprising:
a first beam splitter disposed between the optical fiber bundle and the optical sensing device, and configured to transmit the optical signal from the optical fiber bundle to the optical sensing device; and
a projector configured to convert a second electrical signal relating to a second image to a second optical signal, and transmit the second optical signal to the first beam splitter,
wherein the first beam splitter is further configured to transmit the second optical signal to the optical fiber bundle.

7. The system of claim 6, further comprising:
a second beam splitter disposed between the optical fiber bundle and the optical assembly,
wherein the second beam splitter is configured to transmit the optical signal from the optical assembly to the optical fiber bundle, and transmit the second optical signal from the optical fiber bundle to a display region such that the second image relating to the second optical signal is presented on the display region.

8. The system of claim 7, wherein the display region is in the examination space of the medical device.

9. The system of claim 2, further comprising:
a light source configured to provide a light; and
a fiber optic light guide operably connected to the light source, and transmit the light into the examination space of the medical device.

10. The system of claim 9, wherein the fiber optic light guide and the optical fiber bundle are disposed coaxially or the fiber optic light guide is disposed around the optical fiber bundle.

11. (canceled)

12. The system of claim 9, wherein the light is a structured light having a predefined pattern.

13-16. (canceled)

17. The system of claim 1, further comprising:
a guide member configured to facilitate a position adjustment of the optical assembly; or
a motion determination module configured to determine a motion of the subject based on the monitoring image of the ROI.

18. (canceled)

19. The system of claim 1, wherein the optical assembly includes one or more lenses.

20. A method for monitoring a subject positioned in a medical device, comprising:
obtaining, via an optical assembly, an optical signal associated with a region of interest (ROI) of the subject in an examination space of the medical device;
transmitting, via an optical fiber bundle, the optical signal to an optical sensing device;
converting, via the optical sensing device, the optical signal to an electrical signal; and
generating, via an image generator, a monitoring image of the ROI based on the electrical signal.

21. The method of claim 20, further comprising:
generating, via a light source, a light; and
projecting, via a fiber optic light guide, the light on the ROI of the subject.

22. The method of claim 21, wherein the light is a structured light having a predefined pattern.

23. The method of claim 22, further comprising:
obtaining a plurality of monitoring images at different moments; and
determining a reference image associated with the structured light, and determining a motion of the subject based on the plurality of monitoring images and the reference image; or
determining a motion of the subject based on the plurality of monitoring images.

24. The method of claim 23, further comprising:
obtaining scanning data of the ROI of the subject based on the motion of the subject; and
reconstructing a scanning image based on the scanning data.

25. The method of claim 20, further comprising:
transmitting, via a first beam splitter, the optical signal from the optical fiber bundle to the optical sensing device;
converting, via a projector, a second electrical signal relating to a second image to a second optical signal;
transmitting, via the projector, the second optical signal to the first beam splitter; and
transmitting, via the first beam splitter, the second optical signal to the optical fiber bundle.

26. The method of claim 25, further comprising:
transmitting, via a second beam splitter, the optical signal from the optical assembly to the optical fiber bundle; and
transmitting, via the second beam splitter, the second optical signal from the optical fiber bundle to a display region such that the second image relating to the second optical signal is presented on the display region.

27. A system associated with an imaging device, comprising:
a storage device storing a set of instructions; and
at least one processor in communication with the storage device, wherein when executing the instructions, the at least one processor is configured to cause the imaging system to:
cause an optical sensing device to convert an optical signal associated with a region of interest (ROI) of a subject in an examination space of the medical device to an electrical signal; and
cause an image generator to generate, based on the electrical signal, a monitoring image of the ROI;
wherein the optical signal is obtained according to a process, including,
obtaining, via an optical assembly, the optical signal associated with the ROI; and
transmitting, via an optical fiber bundle, the optical signal to the optical sensing device.

28-31. (canceled)
